(19) United States
(12) Patent Application Publication
(10) Pub. No.: US 2011/0237597 A1
(43) Pub. Date: Sep. 29, 2011

(54) METHOD OF TREATING PERIPHERAL NERVE SENSORY LOSS USING COMPOUNDS HAVING NICOTINIC ACETYLCHOLINE RECEPTOR ACTIVITY

(76) Inventors: Theresa A. Zesiewicz, Tampa, FL (US); Kelly L. Sullivan, Tampa, FL (US); John Edward Ramsey, Forest, VA (US)

(21) Appl. No.: 12/993,457

(22) PCT Filed: May 15, 2009
(86) PCT No.: PCT/US09/44155

§ 371 Date: Jun. 14, 2011

Related U.S. Application Data
(60) Provisional application No. 61/055,692, filed on May 23, 2008.

(51) Int. Cl. A61K 31/4995; A61P 25/02

(52) U.S. Cl. 514/250

(57) ABSTRACT

Methods for treatment of peripheral nerve sensory loss are disclosed. The methods involve treating a patient with a compound having nicotinic acetylcholine receptor activity.

### CROSS-REFERENCE TO RELATED APPLICATIONS

The present application claims the benefit of U.S. Provisional Application Ser. No. 61/055,692, filed May 23, 2008, which is hereby incorporated by reference herein in its entirety.

### BACKGROUND

The present disclosure generally relates to methods for treatment of peripheral nerve sensory loss. These symptoms can be treated in a patient by administering to the patient a compound having nicotinic acetylcholine receptor activity.

Nicotinic acetylcholine receptors are present in many tissues in the body, including, for instance, in peripheral nerves, dorsal root ganglia, and the spinal cord. The stimulation of nicotinic acetylcholine receptors (nAChRs) leads to an antinociceptive effect. Recent evidence has suggested that the anti-allodynic effect of neuronal acetylcholine receptor (nAChR) agonists may have a peripheral component (L. E. Rueter, et al., Pain, 2003 June; 103(3):269-76). There is also evidence that there is loss of functional neuronal nicotinic receptors in dorsal root ganglion neurons in a rat model of neuropathic pain (G. R. Dubé, et al. Neurosci. Lett., 2005 Mar. 7; 376(1):29-34). In the spinal cord, nicotinic receptors are expressed on primary afferent terminals, inhibitory interneurons and descending noradrenergic and serotoninergic fibers. Injury to, or abnomalities of, the spinal cord, dorsal root ganglia, motor neurons, brain, peripheral nerves, or associated structures, in general, may alter the expression of numerous receptors involved in nociceptive processing in the superficial dorsal horn of the spinal cord.

The role of nicotine on the human cerebellum is unclear. Nicotinic receptors appear responsible for disorders like Alzheimer's disease, anxiety, drug addition, epilepsy, Parkinson's Disease, schizophrenia, and Tourette's Syndrome. A report by Pereira et al. (NeuroReport 2001, 8, 1223-1226) showed that nicotine was associated with postural imbalance in non-smokers and occasionally in smokers, and also contributed to nystagmus and body sway (Spillane J D, Br. Med. J. 1955, 2: 1345, 1345-1351). Prenatal or neonatal nicotine exposure is thought to interfere with brain development in both human and animal studies. In fact, nicotine crosses the placenta (Al-Rejaie et al., Alcohol Clin. Exp. Res. 2006, 30(7), 1223-33). Smoking during pregnancy has been associated with miscarriage, sudden infant death syndrome (SIDS), and neurobehavioral disturbances including hyperactivity, depression, and anxiety (Smith et al., Brain Res. 2006, 1115(1), 16-25; Katz et al., J. Physiol. 1967, 138, 63-80). One study found that concurrent exposure of the human brain to alcohol and nicotine during a brain growth spurt reduced the total number of Purkinje cells (Arneric et al., Biochemical Pharmacology 2007, 74, 1092-1101). Another study compared the expression of nicotinic and muscarinic acetylcholine receptors in the first trimester in the pons, medulla oblongata, and cerebellum in 5-12 week gestation abortus of smoking and non-smoking women (Katz et al., J. Physiol. 1967, supra). The gene expression pattern of both α4 and α7 nicotinic receptor subunits in these regions was altered after smoking. These findings suggest that early prenatal nicotine exposure affects the normal developmental pattern of the human fetal cholinergic system.

Unlike nicotine, selective activation of nicotinic acetylcholine receptors, such as the α4β2 nicotinic acetylcholine receptor, may improve ataxia (Al-Rejaie et al., Alcohol Clin. Exp. Res. 2006, supra). Partial agonism of this receptor has been shown to decrease ataxia in animal models that was induced by alcohol (Al-Rejaie et al., Alcohol Clin. Exp. Res. 2006, supra) or tetrahydrocannabinoid (Smith et al., Brain Res. 2006, supra). Nicotinic acetylcholine receptors are rapidly desensitized by up-regulation (Katz et al., J. Physiol. 1967, supra), and partial α₄β₂ nicotinic acetylcholine receptors agonists like varenicline may paradoxically behave as antagonists rather than agonists (Arneric et al., Biochemical Pharmacology 2007, supra).

The α₄β₂ receptor in the mammalian brain has been linked to reward, tolerance and sensitization of nicotine (West et al., Psychopharmacology 2008; 197(3):371-7). In vivo studies of nicotinic acetylcholine receptors found an increased binding of [3H]nicotine in several areas of the brain in smokers, with prominent regional differences of distribution volumes in the cerebellum and brain stem, with an increased uptake in smokers compared to non-smokers (West et al., Psychopharmacology 2008, supra).

Varenicline is a recently-developed drug structurally based on cytisine, used as a prescription drug to combat smoking addition. Varenicline is a nicotinic receptor agonist, acting as a partial agonist of many nicotinic acetylcholine receptors, including the α₄β₂ subtype, found in the cerebellum (Schmitz-Hübsch et al., Neurology 2006, supra). Recent reports also show varenicline acts as a potent, full agonist of the α₇ receptor subtype (K. Minalak, et al., Molec. Pharm., 70(3):801-805 (2006)). As noted above, nicotinic acetylcholine receptors rapidly desensitize by up-regulation of the active agent leading to the hypothesis that certain agents may act on these receptors functionally as antagonists, rather than as agonists.

Multiple neurodegenerative diseases, injuries, and toxic exposures can lead to the progressive loss of the ability to coordinate movements. Symptoms of these physically devastating diseases and conditions include sensory loss. Sensory loss caused by, among other things, cerebellar disease, progressive supranuclear palsy (PSP) and atypical parkinonisms, for instance, currently have no treatment or cure.

### SUMMARY OF THE DISCLOSURE

Among the various aspects of the present invention is the provision of methods for treatment of sensory loss, including peripheral nerve sensory loss, that may result from, for example, injuries to the spinal cord, dorsal root ganglia, motor neurons, brain, peripheral nerves, or associated structures, or diseases or abnormalities relating to these systems and structures.

Briefly, therefore, the present invention is directed in one aspect to a method of treating peripheral nerve sensory loss in a human, the method comprising administering to the human a compound having nicotinic acetylcholine receptor activity.

Another aspect of the invention is directed to a method of treating peripheral nerve sensory loss in a human, the method comprising: determining a baseline measurement of peripheral nerve sensory loss in the human and thereafter administering to the human a compound having nicotinic acetylcholine receptor activity; and determining a second measurement of peripheral nerve sensory loss in the human during or after administration of the compound, wherein an improvement in the second measurement relative to the baseline measurement indicates treatment of the peripheral nerve sensory loss.

Another aspect of the invention is directed to a method of treating peripheral nerve sensory loss in a human, the method comprising: determining a baseline measurement of peripheral nerve sensory loss in the human and thereafter administering to the human a compound having nicotinic acetylcholine receptor activity; and determining a second measurement of peripheral nerve sensory loss in the human at least one month after administration of the compound has ceased; wherein the second measurement is improved relative to the baseline measurement.

Another aspect of the invention is directed to a method of treating peripheral nerve sensory loss in a human, the method comprising administering to the human a compound having nicotinic acetylcholine receptor activity, wherein a second measurement of peripheral nerve sensory loss measured after ceasing administration of the compound is improved relative to a baseline measurement of peripheral nerve sensory loss measured prior to administration of the compound.

Another aspect of the invention is directed to use of a compound having nicotinic acetylcholine receptor activity in the manufacture of a medicament for the treatment of peripheral nerve sensory loss.

Another aspect of the invention is directed to an (i) aryl-fused azapolycyclic compound; (ii) pyridopyranoazepine; (iii) aryl-substituted olefinic amine compound; (iv) benzylidene- or cinnamylidene-anabaseine compound; (v) heterocyclic ether compound; (vi) 3-pyridyloxyalkyl heterocyclic ether compound; (vii) N-substituted diazabicyclic compound; (viii) heterocyclic substituted amino azacycle compound; or (ix) indazole, benzothioazole, or benzoisothiazole compound for use in the therapeutic treatment of peripheral nerve sensory loss.

Another aspect of the invention is directed to an aryl-fused azapolycyclic compound for use in the therapeutic treatment of peripheral nerve sensory loss.

In each of these and other aspects, in one general embodiment the compound may selected from the group consisting of ABT-089, ABT-894, alpha-bungarotoxin, anabaseine, bupropion, buspirone, BW284c51, cytisine, dianicline (SSR591813), dihydro-beta-erythoidine, DMXB, DMXB-A (GTS-21), diazoxon, donepezil, exelon, fluoxetine, galantamine, huperzine A, ispronicline (TC-1734/AZD-3480), lobeline, mecamylamine, MEM3454, MEM63908, methyllycaconitine, nefazodone, octanol/ethanol, OmIA, paroxetine, sertraline, tacrine, TC-2559, TC-5214 ((S)-(+)-mecamylamine), TC-5619, tebanicline (ABT-594), varenicline, venlafaxine, XY4083, and combinations thereof. In one general embodiment, the compound is selected from the group consisting of ABT-089, ABT-894, bupropion, cytisine, dianicline (SSR591813), DMXB-A (GTS-21), ispronicline (TC-1734/AZD-3480), lobeline, mecamylamine, methyllycaconitine, TC-2559, TC-5214 ((S)-(+)-mecamylamine), tebanicline (ABT-594), varenicline, and combinations thereof. In another general embodiment, the compound is selected from the group consisting of donepezil, exelon, fluoxetine, galantamine, huperzine A, MEM3454, MEM63908, tacrine, XY4083, and combinations thereof. In another general embodiment of these and other aspects, the compound is, for example, a smoking cessation agent that operates through nicotinic acetylcholine receptor activity; in one particular embodiment, for example, the compound is varenicline.

Other objects and features will be in part apparent and in part pointed out hereinafter.

### DETAILED DESCRIPTION

The present disclosure provides methods for the treatment of certain sensory loss symptoms in a patient, typically a human. In general, the methods described herein may be utilized to treat peripheral nerve sensory loss, or may be used to treat sensory loss from central mechanisms such as the brain, spinal cord, and associated structures. Regardless of the underlying result, cause, or structure(s) and system(s) involved, the general sensory loss symptoms may involve, by way of example, the loss of prioprioception, the loss of the ability to feel vibration, the loss of position sense, and/or the loss of the ability to feel light and deep touch, among other things.

The disclosure relates, in part, to the discovery that compounds having nicotinic acetylcholine receptor activity (and pharmaceutical compositions including such compounds) may be used to treat peripheral nerve sensory loss. Accordingly, a preferred embodiment of the present disclosure is the use of agents and compounds having nicotinic acetylcholine receptor activity, such as, for example, varenicline, in methods for treating peripheral nerve sensory loss. For example, one embodiment of the present disclosure is directed to a method for treating peripheral nerve sensory loss in a human, the method comprising administering to the human a compound having nicotinic acetylcholine receptor activity. Another embodiment of the present disclosure is directed to a method for treating peripheral nerve sensory loss in a human, the method comprising administering to the human a compound having nicotinic acetylcholine receptor activity. In certain of these and other embodiments, the compound administered to the patient is varenicline.

Generally, the compounds having nicotinic acetylcholine receptor activity may have agonistic, antagonistic, and/or modulatory activity, or any other activity associated with sensory loss. In certain embodiments, the compound having nicotinic acetylcholine receptor activity is a nicotinic acetylcholine receptor agonist. In one preferred embodiment, the compound is varenicline. These and other compounds are used in methods for treating peripheral nerve sensory loss resulting from a range of underlying diseases and medical conditions, including diseases resulting from chronic or long-term exposure to toxins (such as drugs, alcohol, or other agents), and other diseases or conditions that affect the peripheral nervous system, and other central mechanisms such as the brain, spinal cord, dorsal root ganglia, motor neurons, and associated structures. The methods comprise administering to the patient the nicotinic acetylcholine receptor-active compound.

As noted above, the methods disclosed herein involve administering to a patient exhibiting peripheral nerve sensory loss symptoms, resulting from an underlying disease, a compound having nicotinic acetylcholine receptor activity. As noted elsewhere herein, the patient exhibiting sensory loss symptoms may be experiencing one or more of the loss of proprioception, the loss of the ability to feel vibration, the loss of position sense, and the loss of the ability to feel light and deep touch. In certain embodiments, the patient is administered a pharmaceutical composition comprising the compound; in one particular embodiment, the compound is varenicline. A patient receiving such treatment may exhibit substantial improvement relative to a baseline score calculated or determined prior to treatment. The treatments disclosed herein are also capable of providing a neuroprotective or disease-modifying effect; that is, the treatment involves affecting chemical or biochemical changes in the patient that persist even after treatment is stopped. Without being bound to any particular theory, it is believed that, over time, administration of the compound stabilizes the cell membrane of a neuronal cell and/or helps in the normalization of neuronal cell functions (e.g., the maintenance and recovery of such functions).

The discovery that compounds having nicotinic acetylcholine receptor activity can be used to treat peripheral nerve sensory loss symptoms from a wide range of diseases has clinical significance. Nicotinic acetylcholine receptor-active compounds may be used to treat adverse symptoms of peripheral nerve sensory loss associated with, for example, Friedreich's ataxia, among a range of others.

One embodiment of the methods of the present disclosure, therefore, comprises administering to a human in need of such treatment a compound having nicotinic acetylcholine receptor activity, typically in the form of a pharmaceutical composition comprising such compound. The compound is generally administered in an effective amount; that is, a dose of sufficient size to have a detectable therapeutic effect on the patient's peripheral nerve sensory loss symptoms. The therapeutic effect may be, for instance, any treatment that improves a patient's symptoms or otherwise reduces, alleviates, or minimizes such adverse conditions. In general, therefore, the treatment or treating of the symptoms discussed herein (i.e., sensory loss) refer to the improvement, amelioration, reduction, or minimization of these symptoms in an individual. It will be appreciated by the person of ordinary skill in the art that a treatment need not be completely effective in reducing or eliminating the symptom(s). Any reduction in the severity of symptoms or delay in the progression of symptoms is desirable to a patient and thus contemplated in the present disclosure. In this regard, it is noted that the methods described herein are not directed to treatment or prophylaxis of the underlying disease, but rather are directed to improving, ameliorating, reducing, or minimizing the subjective indications that characterize the disease (i.e., the symptoms), including physical and physiological manifestations or reactions, and in particular, peripheral nerve sensory loss. The human patient may be, in various embodiments, an infant, child, adolescent, or adult.

Peripheral Nerve Sensory Loss

As noted above, the present disclosure relates to the treatment of sensory loss, such as peripheral nerve sensory loss. Sensory loss, in general, refers to reduction in or loss of the sense of touch and pressure (light and deep), vibratory sense (pallesthesia), position sense, proprioception, and crude touch, and reduction in appreciation of the spatial qualities of the stimuli. Symptoms of sensory loss often start gradually, then progressively get worse. In general, peripheral nerve sensory loss may be a secondary symptom of diseases or conditions that affect the nervous system (including, for example, posterior columns of the spinal cord, dorsal root ganglion, dorsal column, corticospinal tracts, rubrospinal tract, vestibulospinal tract, spinocerebellar tracts, spinoreticular tracts, alpha motor neurons to muscle fibers and gamma motor neurons to muscles spindles, peripheral nerves, muscles, sympathetic or parasympathetic nervous system, and substantia gelantinosa) or other structure or system. Sensory loss may be caused by sensory abnormalities that arise from the central nervous system including the trigeminal system leading to proprioceptive, vibratory, and position sense loss.

The patient's sensory loss may be disease-induced; that is, it is caused by a disease. Additionally or alternatively, the sensory loss may be non-disease-induced, for example, drug-induced symptoms of sensory loss, e.g., resulting from the immediate exposure to drugs or alcohol or other toxins, or the sensory loss may be induced by acute or traumatic injury such as caused by contusion, laceration, acute spinal cord injury, CNS degeneration, etc. In one embodiment, for example, the peripheral nerve sensory loss is disease-induced. In another embodiment, the peripheral nerve sensory loss is drug- or toxin-induced. In yet another embodiment, the peripheral nerve sensory loss is induced by acute or traumatic injury. Additionally or alternatively, underlying diseases manifesting in peripheral nerve sensory loss may be unknown, thus the peripheral nerve sensory loss may also result from idiopathic cases, including those due to anxiety or aging.

As noted above, peripheral nerve sensory loss may result from a wide range of diseases, disorders, and environmental factors, including, but not limited to, neurodegenerative disorders, Friedreich's ataxia, metabolic disorders, diseases resulting from vitamin deficiencies, trauma, stroke or vascular disease, infection (e.g., epidural abscesses), tuberculosis of the spine, inflammation, meningeal arachnoiditis, transverse myelitis (an acute, usually ascending inflammation of cord, caused by multiple sclerosis, viral infections or SLE), HIV and HTLV 1, tabes dorsalis (syphilis), tumours, metastases (e.g., bronchus, breast and prostate), meningeal infiltration by carcinoma or leukaemia, tumours arising from the dura or meningioma, nerve sheath neurofibroma, diabetes and other endocrine disorders, diseases resulting from chronic exposure to toxins, endocrine disorders, Charcot-Marie-Tooth (CMT) Disease, mitochondrial diseases, myopathies, celiac disease, genetic disorders, Guillain-Barré, ganglionopathies, Varicella Zoster, Herpes, Dysautonomia, demyelinating conditions, cerebellar diseases, thalamic diseases, syrinx, spinal cord injury, and other acquired causes of proprioceptive, position, and vibratory loss in the extremities. Other causes of peripheral nerve sensory loss include autonomic neuropathy, brachial plexus injury (Erb's Palsy), injuries such as burners and stingers, burning feet, cervical radiculopathy, chronic inflammatory demyelinating polyneuropathy (CIDP), diabetic neuropathy, dysautonomia, giant axonal neuropathy, glossopharyngeal neuralgia, hereditary neuropathies, hereditary spastic paraplegia, Isaac's Syndrome, pinched nerve, polyneuropathy, AIDS neuropathy, postherpetic neuralgia, and ulnar nerve entrapment.

Symptoms of peripheral nerve sensory loss may also result from a wide range of disorders of the nervous system, also known as neuropathies, which may include disorders of the peripheral nervous system and the central nervous system. Peripheral nerve disorders, for instance, can affect one nerve or many nerves. Some peripheral nerve disorders, such as diabetic nerve problems, are the result of other diseases, while others, like Guillain-Barré Syndrome, occur after a viral infection. Still other peripheral nerve disorders are caused or exacerbated by nerve compression, such as in carpal tunnel syndrome or thoracic outlet syndrome. In other cases, like complex regional pain syndrome, the symptoms of peripheral nerve sensory loss begin after an injury, e.g., to the brain or spinal cord. In still other cases, the symptoms of peripheral nerve sensory loss are congenital. Examples of neuropathies that may result in symptoms of peripheral nerve sensory loss include amyloid neuropathies, brachial plexus neuropathis, complex regional pain syndromes, mononeuropathies, nerve compression syndromes, neuralgia, neuritis, peripheral nervous system neoplasms, polyneuropathis, acrodynia, hand-arm vibration syndrome, Isaac's Syndrome, neurofibromatosis 1, congenital pain insensitivity, spinal cord injury, and Tarlov cysts.

Nervous system disorders, for instance, can generally be grouped by cause. Genetic causes of peripheral nerve sensory loss include disorders or diseases such as Friedreich's ataxia and Charcot-Marie-Tooth syndrome. Metabolic or endocrine causes of peripheral nerve sensory loss include disorders or diseases such as diabetes mellitus, chronic renal failure, porphyria, amyloidosis, liver failure, and hypothyroidism. Toxic causes of peripheral nerve sensory loss include alcoholism, drug use (including without limitation drugs such as vincristine, phenyloin and isoniazid), organic metals, heavy metals, excess intake of Vitamin B6 (pyridoxine), or fluoroquinolone toxicity. Inflammatory causes of peripheral nerve sensory loss include disorders or diseases such as Guillain-Barré Syndrome, systemic lupus erythematosis, leprosy, and Sjögren's syndrome. Vitamin deficiency states such as a deficiency in Vitamin B12, Vitamin A, Vitamin E, or thiamin (Vitamin B1) may also cause peripheral nerve sensory loss. Physical trauma such as compression, pinching, cutting, projectile injuries (i.e. gunshot wound), or strokes including the prolonged occlusion of blood flow may be a cause of peripheral nerve sensory loss. In addition, other causes of peripheral nerve sensory loss include shingles, malignant disease, HIV (human immunodeficiency virus), radiation, and chemotherapy.

Peripheral neuropathies, for instance, may either be symmetrical and generalized or focal and multifocal, which can be one indicator of the cause of the peripheral nerve disease. Generalized peripheral neuropathies are symmetrical and usually due to various systematic illnesses and disease processes that affect the peripheral nervous system in its entirety. They are further divided into several categories. Distal axonopathies are the result of some metabolic or toxic derangement of neurons. They may be caused by metabolic diseases such as diabetes, renal failure, deficiency syndromes such as malnutrition and alcoholism, or the effects of toxins or drugs. Myelinopathies are due to a primary attack on myelin causing an acute failure of impulse conduction. One common cause is acute inflammatory demyelinating polyneuropathy (AIDP; also known as Guillain-Barré Syndrome), though other causes include chronic inflammatory demyelinating polyneuropathy (CIDP), genetic metabolic disorders (e.g. leukodystrophy), or toxins. Neuronopathies are the result of destruction of peripheral nervous system and other neurons. They may be caused by motor neuron diseases, sensory neuropathies (e.g. Herpes zoster), toxins or autonomic dysfunction. Neurotoxins may cause neuronopathies, such as the chemotherapy agent vincristine. Diseases that affect the Dorsal Root Ganglia and motor neurons include without limitation Fabry's Disease, Nieman-Pick disease, and “Anterior Horn Diseases” namely Amyotrophic Lateral Sclerosis, spinal muscular atrophy, Charcot-Marie-Tooth disease, poliomyelitis, progressive muscular atrophy, spinal and bulbar muscular atrophy (Kennedy disease), and paraneoplastic conditions.

Compounds Having Nicotinic Acetylcholine Receptor Activity

Compounds for treating peripheral nerve sensory loss symptoms according to the methods described herein have nicotinic acetylcholine receptor activity. As noted above, this activity may be agonistic, antagonistic, or modulatory. The compound(s) may have an effect on either the neuronal type nicotinic acetylcholine receptors, the muscle type nicotinic acetylcholine receptor, or both. For example, the compound may be capable of acting on the α1, β1, δ, γ, and ε receptor subunits, and combinations thereof. By way of another example, the compound may be capable of acting on the various homomeric or heteromeric combinations of seventeen different nicotinic receptor subunits: α2 through α10 and β2 through β4 (e.g., the neuronal subtypes: (α4)₃(β2)₂, (α4)₂(β2)₃, and (α7)₅). Generally speaking, this includes, for instance, compounds having activity on Neuronal Type I receptor subunits (e.g., α9, α10), Neuronal Type II receptor subunits (e.g., α7, α8), Neuronal Type III(1) receptor subunits (e.g., α2, α3, α4, and α6), Neuronal Type III(2) receptor subunits (e.g., β2, β4), Neuronal Type III(3) receptor subunits (e.g., β3, β5), Muscle Type IV receptor subunits (e.g., α1, β1, δ, γ, and ε), and combinations thereof.

For example, the compound may be an agonist or partial agonist (including selective agonist or selective partial agonist) of the α4β2 receptor (e.g., ABT-089, ABT-894, cytosine, dianicline (SSR591813), TC-1734, TC-2559, and varenicline, among others). In additional or alternative examples, the compound may be an antagonist of the α4β2 receptor (e.g., anabaseine, DMXB-A, lobeline, mecamylamine, methyllycaconitine, and TC-5214, among others). In another example, the compound may be an antagonist (including non-competitive antagonists) of the α3β2 receptor (e.g., alpha-bungarotoxin, bupropion, fluoxetine, lobeline, and mecamylamine, among others). By way of another example, the compound may be an agonist (including selective agonists) of the α7 receptor (e.g., anabaseine, DMXB-A, galantamine, MEM3454, MEM63908, TC-5214, and varenicline, among others). In another example, the compound may be an antagonist of the α7 receptor (e.g., alpha-bungarotoxin, dihydro-beta-erythroidine, mecamylamine, paroxetine, sertraline, and venlafaxine, among others). In another example, the compound may be an antagonist of the α3β4 receptor (e.g., alpha-bungarotoxin, bupropion, fluoxetine, lobeline, and mecamylamine, among others). By way of another example, the compound may be an antagonist of the α3β4 receptor (e.g., fluoxetine, nefazodone, paroxetine, sertraline, and venlafaxine, among others). By way of yet another example, the compound may have activity (e.g., agonistic, antagonistic, or other activity) on the α3β2, α6, β2, (α1)2β1δε and (α1)2β1δγ, γ3, and/or α6β2 receptors (e.g., varenicline, cytosine, alpha-bungarotoxin, ABT-594, and OmIA, among others).

In some instances, the active agent having nicotinic acetylcholine receptor activity is a known compound with proven clinical efficacy, for example, in smoking cessation. In certain embodiments, the compound is selected from the group consisting of (i) an aryl-fused azapolycyclic compound; (ii) a pyridopyranoazepine; (iii) an aryl-substituted olefinic amine compound; (iv) a benzylidene- or cinnamylidene-anabaseine compound; (v) a heterocyclic ether compound; (vi) 3-pyridyloxyalkyl heterocyclic ether compound; (vii) an N-substituted diazabicyclic compound; (viii) a heterocyclic substituted amino azacycle compound; and (ix) an indazole, benzothioazole, or benzoisothiazole compound. In one embodiment, the compound is selected from the group consisting of ABT-089, ABT-894, alpha-bungarotoxin, anabaseine, bupropion, buspirone, BW284c51, cytisine, dianicline (SSR591813), dihydro-beta-erythoidine, DMXB, DMXB-A (GTS-21), diazoxon, donepezil, exelon, fluoxetine, galantamine, huperzine A, ispronicline (TC-1734/AZD-3480), lobeline, mecamylamine, MEM3454, MEM63908, methyllycaconitine, nefazodone, octanol/ethanol, OmIA, paroxetine, sertraline, tacrine, TC-2559, TC-5214 ((S)-(+)-mecamylamine), TC-5619, tebanicline (ABT-594), varenicline, venlafaxine, XY4083, and combinations thereof. In particular embodiment, the compound is selected from the group consisting of ABT-089, ABT-894, bupropion, cytisine, dianicline (SSR591813), DMXB-A (GTS-21), ispronicline (TC-1734/AZD-3480), lobeline, mecamylamine, methyllycaconitine, TC-2559, TC-5214 ((S)-(+)-mecamylamine), tebanicline (ABT-594), varenicline, and combinations thereof. In another particular embodiment, the compound is selected from the group consisting of donepezil, exelon, fluoxetine, galantamine, huperzine A, MEM3454, MEM63908, tacrine, XY4083, and combinations thereof. In a preferred embodiment, the compound is selected from the group consisting of varenicline, dianicline, ispronicline, and combinations thereof; more preferably in this embodiment, the compound is varenicline.

In one particular embodiment, the compound is an aryl-fused azapolycyclic compound. According to this embodiment, for example, the compound administered to the patient may have the formula (i):

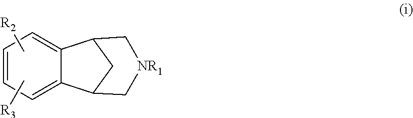

wherein R₁ is hydrogen, (C₁-C₆)alkyl, unconjugated (C₃-C₆)alkenyl, benzyl, XC(═O)R₁₃ or —CH₂CH₂—O—(C₁-C₄)alkyl;

R₂ and R₃ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino-, [(C₁-C₆)alkyl]₂amino-, —CO₂R₄, —CONR₅R₆, —SO₂NR₇R₈, —C(═O)R₁₃, —XC(═O)R₁₃, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur, and X₂(C₀-C₆)alkoxy-(C₀-C₆)alkyl-, wherein X₂ is absent or X₂ is (C₁-C₆)alkylamino- or [(C₁-C₆)alkyl]₂amino-, and wherein the (C₀-C₆)alkoxy-(C₀-C₆)alkyl- moiety of said X₂(C₀-C₆)alkoxy-(C₀-C₆)alkyl- contains at least one carbon atom, and wherein from one to three of the carbon atoms of said (C₀-C₆)alkoxy-(C₀-C₆)alkyl- moiety may optionally be replaced by an oxygen, nitrogen or sulfur atom, with the proviso that any two such heteroatoms must be separated by at least two carbon atoms, and wherein any of the alkyl moieties of said (C₀-C₆)alkoxy-(C₀-C₆)alkyl- may be optionally substituted with from two to seven fluonne atoms, and wherein one of the carbon atoms of each of the alkyl moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluonne atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)—, [(C₁-C₆)alkyl]₂amino-, —CO₂R₄, —CONR₅R₆, —SO₂NR₇R₈, —C(═O)R₁₃ and —XC(═O)R₁₃; or R₂ and R₃, together with the carbons to which they are attached, form a four to seven membered monocyclic, or a ten to fourteen membered bicyclic, carbocyclic ring that can be saturated or unsaturated, wherein from one to three of the nonfused carbon atoms of said monocyclic rings, and from one to five of the carbon atoms of said bicyclic rings that are not part of the benzo ring shown in formula (i), may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂amino-, —CO₂R₄, —CONR₅R₆, —SO₂NR₇R₈, —C(═O)R₁₃, and —XC(═O)R₁₃;

each R₄, R₅, R₆, R₇, R₈ and R₁₃ is selected, independently, from hydrogen and (C₁-C₆) alkyl, or R₅ and R₆, or R₇ and R₈ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and each X is, independently, (C₁-C₆)alkylene: with the proviso that: (a) at least one of R₁, R₂ and R₃ must be the other than hydrogen, and (b) when R₂ and R₃ are hydrogen, R₁ cannot be methyl or hydrogen; and the pharmaceutically acceptable salts of such compounds.

In a particular embodiment, R₁, R₂, and R₃ are each hydrogen; more preferably in this embodiment, the compound has the formula:

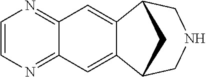

This compound 7,8,9,10-tetrahydro-6,10-methano-6H-pyrazino (2,3-h)(3) benzazepine (also known as varenicline or Chantix®) is approved as a medication for the treatment nicotine dependence. Compounds corresponding to formula (i) and varenicline are described in further detail in International Publication No. WO2001/062736; U.S. Pat. No. 6,410,550; U.S. Pat. No. 6,605,610; U.S. Pat. No. 6,890,927; and U.S. Pat. No. 7,265,119 (each of which is hereby incorporated by reference herein in its entirety). In a particular embodiment, the compound administered to the patient is varenicline.

In another particular embodiment, the compound is a pyridopyranoazepine. According to this embodiment, for example, the compound administered to the patient may have the formula (ii):

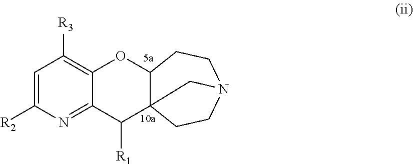

wherein R₁ is a hydrogen atom, a (C₁-C₄)alkyl group, a phenyl(C₁-C₄)alkyl group, a phenylhydroxy(C₁-C₄)alkyl group, a furanyl(C₁-C₄)alkyl group, or a furanyl-hydroxy(C₁-C₄)alkyl group, R₂ is either a hydrogen or halogen atom or a trifluoromethyl, cyano, hydroxyl, nitro, acetyl, (C₁-C₆)alkyl or (C₁-C₆)alkoxy group or a group of general formula NR₄R₅ in which R₄ is a hydrogen atom or a (C₁-C₄)alkyl or (C₁-C₄)alkanoyl group and R₅ is a hydrogen atom or a (C₁-C₄)alkyl group, or else R₄ and R₅ form, with the nitrogen atom which carries them, a C₄-C₇ ring, or a phenyl or naphthyl group optionally substituted by a halogen atom or a trifluoromethyl, trifluoromethoxy, cyano, hydroxyl, nitro, acetyl, (C₁-C₆)alkyl, (C₁-C₆)alkoxy or methylenedioxy group linked in the 2 and 3 positions of the phenyl ring, and R₃ is a hydrogen or halogen atom or a (C₁-C₄)alkyl group.

The compounds of general formula (ii) can exist in the state of bases or of addition salts to acids. In addition, the atoms in positions 5a and 10a being asymmetric, a compound can exist in the form of pure geometric and optical isomers or of mixtures of the latter. In a particular embodiment, R₁, R₂, and R₃ are each hydrogen; more preferably in this embodiment, the compound has the formula:

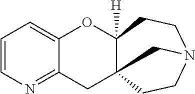

This compound (5aS,8S,10aR)-5a,6,9,10-Tetrahydro, 7H,11H-8,10a-methanopyrido[2′,3′:5,6]pyrano[2,3-d]azepine (also known as SSR-591,813 or dianicline) is presently in clinical trials as a medication for the treatment of nicotine dependence. Compounds corresponding to formula (ii) and dianicline (SSR591813) are described in further detail in U.S. Pat. No. 6,538,003 (hereby incorporated by reference herein in its entirety).

In another particular embodiment, the compound is an aryl-substituted olefinic amine compound. According to this embodiment, for example, the compound administered to the patient may have the formula (iii):

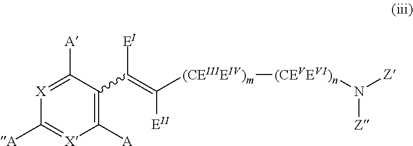

where each of X and X′ are individually nitrogen or carbon bonded to a substituent species characterized as having a sigma m value greater than 0, often greater than 0.1, and generally greater than 0.2, and even greater than 0.3; less than 0 and generally less than −0.1; or 0; as determined in accordance with Hansch et al., Chem. Rev. 91:165 (1991); m is an integer and n is an integer such that the sum of m plus n is 1, 2, 3, 4, 5, 6, 7, or 8, preferably is 1, 2, or 3, and most preferably is 2 or 3; the wavy line in the structure indicates that the compound can have the cis (Z) or trans (E) form; E^{I}, E^{II}, E^{III}, E^{IV}, E^{V} and E^{VI} individually represent hydrogen or lower alkyl (e.g., straight chain or branched alkyl including C₁-C₈, preferably C₁-C₅, such as methyl, ethyl, or isopropyl) or halo substituted lower alkyl (e.g., straight chain or branched alkyl including C₁-C₈, preferably C₁-C₅, such as trifluoromethyl or trichloromethyl), and at least one of E^{I}, E^{II}, E^{III}, E^{IV}, E^{V} and E^{VI} is non-hydrogen and the remaining E^{I}, E^{II}, E^{III}, E^{IV}, E^{V} and E^{VI} are hydrogen; and Z′ and Z″ individually represent hydrogen or lower alkyl (e.g., straight chain or branched alkyl including C₁-C₈ preferably C₁-C₅, such as methyl, ethyl, or isopropyl), and preferably at least one of Z and Z″ is hydrogen, and most preferably Z′ is hydrogen and Z″ is methyl; alternatively Z′ is hydrogen and Z″ represents a ring structure (cycloalkyl or aromatic), such as cyclopropyl, cyclobutyl, cyclopentyl, cyclohexyl, cycloheptyl, adamantyl, quinuclidinyl, pyridyl, quinolinyl, pyrimidinyl, phenyl, benzyl (where any of the foregoing can be suitably substituted with at least one substituent group, such as alkyl, halo, or amino substituents); alternatively Z′, Z″, and the associated nitrogen atom can form a ring structure such as aziridinyl, azetidinyl, pyrollidinyl, piperidinyl, quinuclidinyl, piperazinyl, or morpholinyl.

Representative compounds having the generic structure (iii) include (4E)-N-methyl-5-(3-pyridyl)-4-pen-ten-2-amine, (4E)-N-methyl-5-(5-pyrimidinyl)-4-penten-2-amine, (4E)-N-methyl-5-(5-methoxy-3-pyridyl)-4-penten-2-amine, (4E)-N-methyl-5-(6-amino-5-methyl-3-pyridyl)-4-penten-2-amine, (2R)-(4E)-N-methyl-5-(3-pyridyl)-4-penten-2-amine, (2R)-(4E)-N-methyl-5-(5-isopropoxy-3-pyridyl)-4-penten-2-amine, (4E)-N-methyl-5-(5-bromo-3-pyridyl)-4-penten-2-amine, (4E)-N-methyl-5-(5-ethoxy-3-pyridyl)-4-penten-2-amine, (2S)-(4E)-N-methyl-5-(3-pyridyl)-4-penten-2-amine, (4E)-N-methyl-5-(5-isopropoxy-3-pyridyl)-4-penten-2-amine and (2S)-(4E)-N-methyl-5-(5-isopropoxy-3-pyridyl)-4-penten-2-amine (also known as ispronicline, TC-1734, or AZD-3480). Compounds corresponding to formula (iii) are described in further detail in International Publication WO 99/65876 and WO 00/75710; U.S. Patent Application Publication 2002/0052497; U.S. Pat. No. 6,979,695; and U.S. Pat. No. 7,045,538 (each of which is hereby incorporated by reference herein in its entirety).

In another particular embodiment, the compound is a benzylidene- or cinnamylidene-anabaseine compound. According to this embodiment, for example, the compound administered to the patient may have the formula (iv):

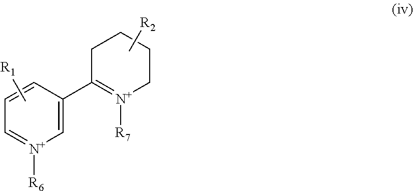

or a salt thereof, wherein R₁, R₆ and R₇ are hydrogen or C₁-C₄ alkyl; and R₂ is ═CHCH═CHX, wherein X is

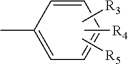

wherein R₃, R₄, and R₅ are selected from the group consisting of hydrogen, C₁-C₄ alkyl optionally substituted with N,N-dialkylamino having 1 to 4 carbon atoms in each of the alkyls, C₁-C₆ alkoxy optionally substituted with N,N-dialkylamino having 1 to 4 carbons in each of the alkyls, carboalkoxy having 1 to 4 carbons in the alkoxy (such as acetoxy), amino, amido having 1 to 4 carbons in the acyl (such as acetylamino), cyano, N,N-dialkylamino having 1 to 4 carbons in each of the alkyls, halo, hydroxyl, and nitro.

Representative cinnamylidene-anabaseines having the generic structure (iv) include, but are not limited to, 3-(4-acetylaminocinnamylidene) anabaseine, 3-(4-hydroxycinnamylidene) anabaseine, 3-(4-methoxycinnamylidene) anabaseine, 3-(4-hydroxy-2-methoxycinnamylidene)anabaseine, 3-(2,4-dimethoxycinnamylidene) anabaseine, and 3-(4-acetoxycinnamylidene) anabaseine. Representative benzylidene-anabaseines having the generic structure (iv) include, but are not limited to, 3-(2,4-dimethoxybenzylidene) anabaseine (also known as DMXB-A and GTS-21), 3-(4-hydroxybenzylidene) anabaseine, 3-(4-methoxybenzylidene) anabaseine, 3-(4-aminobenzylidene) anabaseine, 3-(4-hydroxy-2-methoxybenzylidene) anabaseine, 3-(2-hydroxy-4-methoxybenzylidene) anabaseine, 3-(4-isopropoxybenzylidene) anabaseine, and (7′-methyl-3-(2,4-dimethoxybenzylidene)). Compounds corresponding to formula (iv) are described in further detail in International Publication WO 99/10338 and WO 2006/133303; U.S. Pat. No. 5,741,802; and U.S. Pat. No. 5,977,144 (each of which is hereby incorporated by reference herein in its entirety).

In another particular embodiment, the compound is a heterocyclic ether compound. According to this embodiment, for example, the compound administered to the patient may have the formula (v):

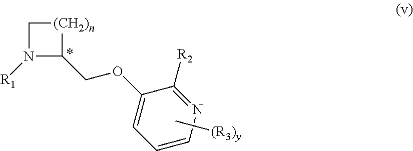

wherein the asterisk indicates a chiral center; n is 1, 2, or 3; y is 1 or 2; R₁ is H, allyl or C₁-C₆-alkyl; R₂ is H, F, Cl, or C₁-C₃-alkyl; and R₃ is independently selected from H, F, Cl, Br or C₁-C₆-alkyl; with the provisos that (a) when R₂ is C₁-C₃-alkyl, then R₁ is H, and (b) when y is 2, then R₂ is hydrogen.

Representative heterocyclic ethers having the generic structure (v) include, but are not limited to, 3-(2-(S)-azetidinylmethoxy)pyridine; 3-((1-methyl-2-S)-azetidinyl)methoxy)pyridine; 2-methyl-3-(2-(S)-azetidinylmethoxy)pyridine (also known as ABT-089); 5-chloro-3-(2-(S)-azetidinylmethoxy)pyridine; 5-([(2R)-azetidin-2-yl]methoxy)-2-chloropyridine (also known as tebanicline or ABT-594); 6-methyl-3-(2-(S)-azetidinylmethoxy)pyridine; 3-(2-(S)-azetidinylmethoxy)chloropyridine; 3-(2-(R)-azetidinylmethoxy)pyridine; 3-((1-methyl-2-(R)-azetidinyl)methoxy)pyridine; 3-(2-(S)-azetidinylmethoxy)-5-bromopyridine; 3-((1-methyl-2-(S)-azetidinyl)methoxy)-5-bromopyridine; and 5,6-dichloro-3-(2-(S)-azetidinylmethoxy)pyridine; 3-(2-(R)-pyrrolidinylmethoxy)pyridine; 3-(2-(S)-pyrrolidinylmethoxy)pyridine; 5-chloro-3-(2-(S)-pyrrolidinylmethoxy)pyridine; 2-methyl-3-(2-(S)-pyrrolidinylmethoxy)pyridine; 6-methyl-3-(2-(S)-pyrrolidinylmethoxy)pyridine; 5-chloro-3-(2-(R)-pyrrolidinylmethoxy)pyridine; 6-methyl-3-(2-(R)-pyrrolidinylmethoxy)pyridine; 3-(2-(S)-pyrrolidinylmethoxy)-6-chloropyridine; 5-bromo-3-(2-(S)-pyrrolidinylmethoxy)pyridine; 3-((1-methyl-2-(S)-pyrrolidinyl)methoxy)pyridine; 5-chloro-3-(1-methyl-2-(S)-pyrrolidinyl)methoxy)pyridine; 6-methyl-3-((1-methyl-2-(S)-pyrrolidinyl)methoxy)pyridine; 5-bromo-3-((1-methyl-2-(S)-pyrrolidinyl)methoxy)pyridine; 6-chloro3-((1-methyl-2-(S)-pyrrolidinyl)methoxy)pyridine; 5-n-butyl-3-((1-methyl-2-(S)-pyrrolidinyl)methoxy)pyridine; 5-n-propyl-3-((1-methyl-2-(S)-pyrrolidinyl)methoxy)pyridine; 5-methyl-3-(1-methyl-2-(S)-pyrrolidinylmethoxy)pyridine; and 5-ethyl-3-(1-methyl-2-(S)-pyrrolidinylmethoxy)pyridine; or a pharmaceutically-acceptable salt or prodrug thereof. Compounds corresponding to formula (v) are described in further detail in International Publication WO 99/32480; U.S. Pat. No. 5,914,328; and U.S. Pat. No. 5,948,793 (each of which is hereby incorporated by reference herein in its entirety).

In another particular embodiment, the compound is a 3-pyridyloxyalkyl heterocyclic ether compound. According to this embodiment, for example, the compound administered to the patient may have the formula (vi):

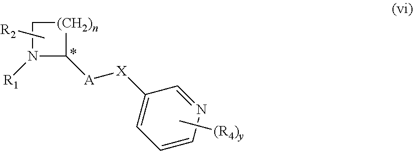

wherein the asterisk indicates a chiral center; n is an integer selected from 1, 2, or 3; X is oxygen or sulfur; R₁ is H, allyl or C₁-C₆-alkyl; R₂ is hydrogen, or when n=2, is a single substituent selected from the group consisting of —CH₂OH, —CH₂F, —CH₂CN, —CH₂OCH₃, —Br, —Cl, —F, —OH, —CH, —(C₁-C₃ alkoxyl), —OCOCH₃, and O-methanesulfonyl, with the proviso that when R₂ is substituted at the 3-position or the 5-position of the pyrrolidinyl ring, it is a C₁-C₃ group; A is selected from the group consisting of:

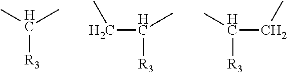

where R₃ is H or C₁-C₆ alkyl; y is 1, 2, or 3 with the provisos that a) when y=1, R₄ is selected from the group consisting of (i) a single substituent at the 2-position of the pyridine ring selected from chlorine and fluorine, and (ii) a single substituent substituted at the 5- or 6-position of the pyridine ring selected from the group consisting of —CN, —CF₃, —NO₂, —CH₂OH, —CH₂CN, —NH₂, —NH—CHO, —NHCO(C₁-C₃ alkyl), —N(C₁-C₃ alkyl)-CO(C₁-C₃ alkyl), —NH—(C1-C₃ alkyl), —N(C₁-C₃ alkyl)₂, —COOH, —COO(C₁-C₃ alkyl), —CONH₂, —CONH(C₁-C₃ alkyl), —CONHbenzyl, and —OCO(C₁-C₃-alkyl); b) when y=2, R⁴ is substituted at the 2,5-, 2,6- or 5,6-positions of the pyridine ring wherein the 2-position substituent is selected from the group consisting of —Br, —Cl, —F, —OH, —(C₁-C₄ alkyl) and —(C₁-C₃ alkoxy) and the substituents at the 5- or 6-positions of the pyridine ring are selected from the group consisting of —Br, —Cl, —F, —OH, —(C₁-C₄ alkyl), —CN, —CF₃, —NO₂, —CH₂OH, —CH₂CN, —(C₁-C₃ alkoxy), —NH₂, —NH—CHO, —NHCO(C₁-C₃ alkyl), —N(C₁-C₃ alkyl)CO(C₁-C₃ alkyl), —NH—(C₁-C₃ alkyl), —N(C₁-C₃alkyl)₂, —COOH, —COO(C₁-C₃ alkyl), —CONH₂, —CONH—(C₁-C₃ alkyl), —CONHbenzyl, and —OCO(C₁-C₃ alkyl); and c) when y=3, R₄ is a substituent at the 2-position of the pyridine ring selected from the group consisting of —Br, —Cl, —F, —OH, —C₁-C₄ alkyl, and —C₁-C₃ alkoxy; and second and third substituents at the 5- and 6-position of the pyridine ring are independently selected from the group consisting of —Br, —Cl, —F, —OH, —C₁-C₄ alkyl, —CN, —CF₃, —NO₂, —CH₂OH, —CH₂CN, —(C₁-C₃ alkoxy), —NH₂, —NH—CHO, —NHCO(C₁-C₃ alkyl), —N(C₁-C₃ alkyl)CO(C₁-C₃ alkyl), —NH—(C₁-C₃ alkyl), —N(C₁-C₃-alkyl)₂, —COOH, —COO(C₁-C₃— alkyl), —CONH₂, —CONH(C₁-C₃ alkyl), —CONHbenzyl, and —OCO(C₁-Cₛ alkyl).

Compounds corresponding to formula (vi) are described in further detail in International Publication WO 96/040682 (hereby incorporated by reference herein in its entirety).

In another particular embodiment, the compound is a N-substituted diazabicyclic compound. According to this embodiment, for example, the compound administered to the patient may have the formula (vii):

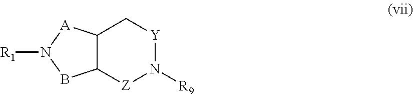

or pharmaceutically acceptable salts and prodrugs thereof, wherein A is selected from the group consisting of a covalent bond, CH₂, CH₂CH₂, and CH₂CH₂CH₂; B is selected from the group consisting of CH₂ and CH₂CH₂, provided that when A is CH₂CH₂CH₂, then B is CH₂; Y is selected from the group consisting of a covalent bond, CH₂, and CH₂CH₂; Z is selected from the group consisting of a covalent bond, CH₂, and CH₂CH₂, provided that when Y is CH₂CH₂, then Z is a covalent bond and further provided that when Z is CH₂CH₂, then Y is a covalent bond;

R₁ is selected from the group consisting of:

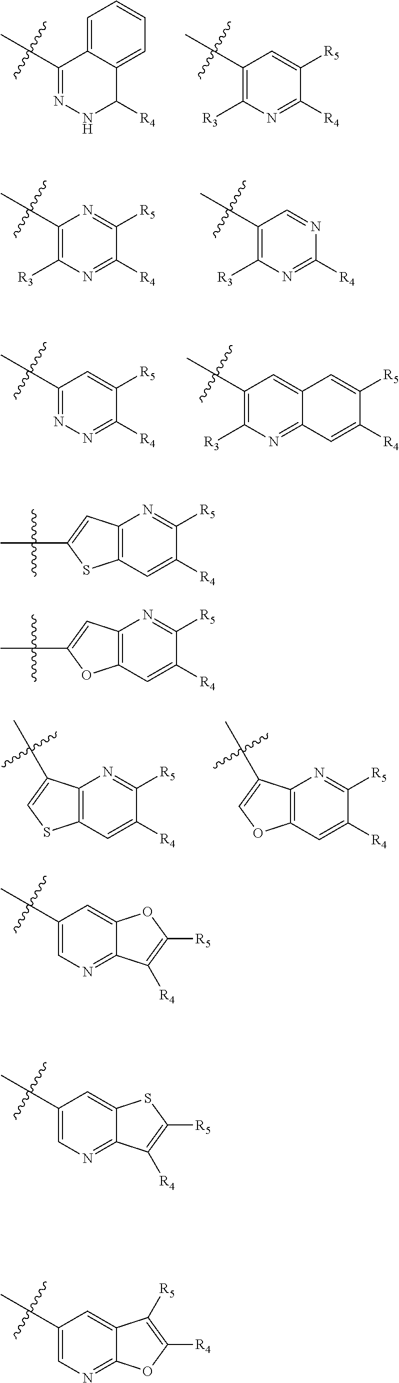

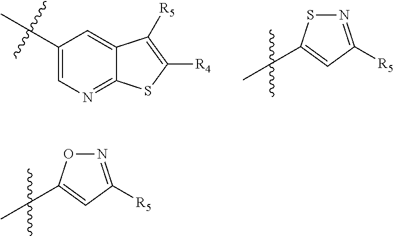

R₃ is selected from the group consisting of hydrogen, alkyl, and halogen;

R₄ is selected from the group consisting of hydrogen, alkoxy, alkyl, amino, halogen, and nitro;

R₅ is selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, amino, aminoalkyl, aminocarbonyl, aminocarbonylalkyl, aminosulfonyl, carboxy, carboxyalkyl, cyano, cyanoalkyl, formyl, formylalkyl, haloalkoxy, haloalkyl, halogen, hydroxy, hydroxyalkyl, mercapto, mercaptoalkyl, nitro, 5-tetrazolyl, —NR₆S(O)₂R₇, —C(NR₆)NR₇R₈, —CH₂C(NR₆)NR₇R₈, —C(NOR₆)R₇, —C(NCN)R₆, —C(NNR₆R₇)R₈, —S(O)₂OR₆, and —S(O)₂R₆;

R₆, R₇, and R₈ are independently selected from the group consisting of hydrogen and alkyl;

and R₉ is selected from the group consisting of hydrogen, alkoxycarbonyl, alkyl, amino, aminoalkyl, aminocarbonylalkyl, benzyloxycarbonyl, cyanoalkyl, dihydro-3-pyridinylcarbonyl, hydroxy, hydroxyalkyl, and phenoxycarbonyl.

Representative heterocyclic ethers having the generic structure (vii) include, but are not limited to, (1R,5R)-6-(6-chloro-3-pyridinyl)-2,6-diazabicyclo[3.2.0]heptane; (1R,5R)-6-(3-pyridinyl)-2,6-diazabicyclo[3.2.0]heptane; (cis)-6-(3-pyridinyl)-3,6-diazabicyclo[3.2.0]heptane; (cis)-6-(6-chloro-3-pyridinyl)-3,6-diazabicyclo[3.2.0]heptane; (1R,5S)-6-(3-pyridinyl)-3,6-diazabicyclo[3.2.0]heptane; (1R,5S)-6-(5-bromo-3-pyridinyl)-3,6-diazabicyclo[3.2.0]heptane; (1S,5R)-6-(6-chloro-3-pyridinyl)-3,6-diazabicyclo[3.2.0]heptane; (1S,5R)-6-(3-pyridinyl)-3,6diazabicyclo[3.2.0]heptane; (1R,5S)-6-(6-chloro-3-pyridinyl)-3,6-diazabicyclo[3.2.0]heptane; (1S,5R)-6-(5-ethynyl-3-pyridinyl)-3,6-diazabicyclo[3.2.0]heptane; (1S,5R)-6-(5-vinyl-3-pyridinyl)-3,6-diazabicyclo[3.2.0]heptane; 5-[(1S,5R)-3,6-diazabicyclo[3.2.0]hept-6-yl]nicotinonitrile; (1S,5R)-6-(5-bromo-3-pyridinyl)-3,6-diazabicyclo[3.2.0]heptane; (1S,5R)-6-(6-bromo-5-vinyl-3-pyridinyl)-3,6-diazabicyclo[3.2.0]heptane; 2-bromo-5-[(1R,5S)-3,6-diazabicyclo[3.2.0]hept-6-yl]nicotinonitrile; (1R,5S)-6-(5-ethynyl-3-pyridinyl)-3,6-diazabicyclo[3.2.0]heptane; 5-[(1R,5S)-3,6-diazabicyclo[3.2.0]hept-6-yl]nicotinonitrile; (cis)-8-(3-pyridinyl)-3,8-diazabicyclo[4.2.0]octane; (cis)-8-(6-chloro-3-pyridinyl)-3,8-diazabicyclo[4.2.0]octane; (1S,6R) (cis)-8-(6-chloro-3-pyridinyl)-3,8-diazabicyclo[4.2.0]octane; (−) (cis)-8-(6-chloro-3-pyridinyl)-3,8-diazabicyclo[4.2.0]octane; 5-[(1R,6S)-3,8-diazabicyclo[4.2.0]oct-8-yl]nicotinonitrile; (1S,6R)-5-[3,8-diazabicyclo[4.2.0]oct-8-yl]nicotinonitrile; (1R,5S)-6-(5,6-dichloro-3-pyridinyl)-3,6-diazabicyclo[3.2.0]heptane; (1S,5R)-6-(5,6-dichloro-3-pyridinyl)-3,6-diazabicyclo[3.2.0]heptane; (cis)-6-(5,6-dichloro-3-pyridinyl)-3,6-diazabicyclo[3.2.0]heptane; (cis)-8-(5-methoxy-3-pyridinyl)-3,8-diazabicyclo[4.2.0]octane; (1R,5S)-6-(5-methoxy-3-pyridinyl)-3,6-diazabicyclo[3.2.0]heptane; (1S,5R)-6-(5-methoxy-3-pyridinyl)-3,6-diazabicyclo[3.2.0]heptane; (cis)-6-(6-bromo-5-methoxy-3-pyridinyl)-3,6diazabicyclo[3.2.0]heptane; (1R,5S)-6-(6-chloro-5-methyl-3-pyridinyl)-3,6-diazabicyclo[3.2.0]heptane; (1S,5R)-6-(6-chloro-5-methyl-3-pyridinyl)-3,6-diazabicyclo[3.2.0]heptane; (1S,6R) (cis)-8-(5-methoxy-3-pyridinyl)-3,8-diazabicyclo[4.2.0]octane; (1R,6S)-8-(5-methoxy-3-pyridinyl)-3,8-diazabicyclo[4.2.0]octane; (cis)-8-(6-chloro-5-methyl-3-pyridinyl)-3,8-diazabicyclo[4.2.0]octane; (1S,6R)-8-(6-chloro-5-methyl-3-pyridinyl)-3,8-diazabicyclo[4.2.0]octane; (1R,6S)-8-(6-chloro-5-methyl-3-pyridinyl)-3,8-diazabicyclo[4.2.0]octane; (1S,6R)-8-(3-pyridinyl)-3,8-diazabicyclo[4.2.0]octane; (1R,6S)-8(3-pyridinyl)-3,8-diazabicyclo[4.2.0]octane; (cis)-8-(5,6-dichloro-3-pyridinyl)-3,8-diazabicyclo[4.2.0]octane (1S,6R)-8-(5,6-dichloro-3-pyridinyl)-3,8-diazabicyclo[4.2.0]octane; (1R,6S)-8-(5,6-dichloro-3-pyridinyl)-3,8-diazabicyclo[4.2.0]octane; (cis)-6-(6-bromo-5-methoxy-3-pyridinyl)-3,6-diazabicyco[3.2.0]heptane; (1R,5S)-6-(6-bromo-5-methoxy-3-pyridinyl)-3,6-diazabicyclo[3.2.0]heptane; (1S,5R)-6-(6-bromo-5-methoxy-3-pyridinyl)-3,6-diazabicyclo[3.2.0]heptane; (cis)-6-(5-azido-3-pyridinyl)-3,6-diazabicylo[3.2.0]heptane; (1R,5S)-6-(5-azido-3-pyridinyl)-3,6-diazabicyclo[3.2.0]heptane; and (1R,5S)-6-(5-azido-3-pyridinyl)-3,6-diazabicyclo[3.2.0]heptane. Compounds corresponding to formula (vii) are described in further detail in International Publication WO 2004/0186107; and U.S. Pat. No. 6,809,105 (each of which is hereby incorporated by reference herein in its entirety).

In another particular embodiment, the compound is a heterocyclic substituted amino azacycle compound. According to this embodiment, for example, the compound administered to the patient may have the formula (viii):

Z—R₃ (viii)

wherein Z is selected from the group consisting of:

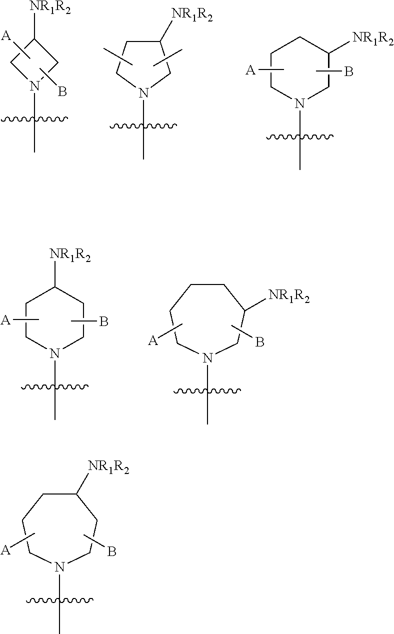

R₁ and R₂ are independently selected from the group consisting of hydrogen and alkyl; A and B are independently absent or independently selected from the group consisting of alkenyl, alkoxy, alkoxycarbonyl, alkyl, alkynyl, carboxy, haloalkyl, halogen, hydroxyl, and hydroxyalkyl; R₃ is selected from the group consisting of:

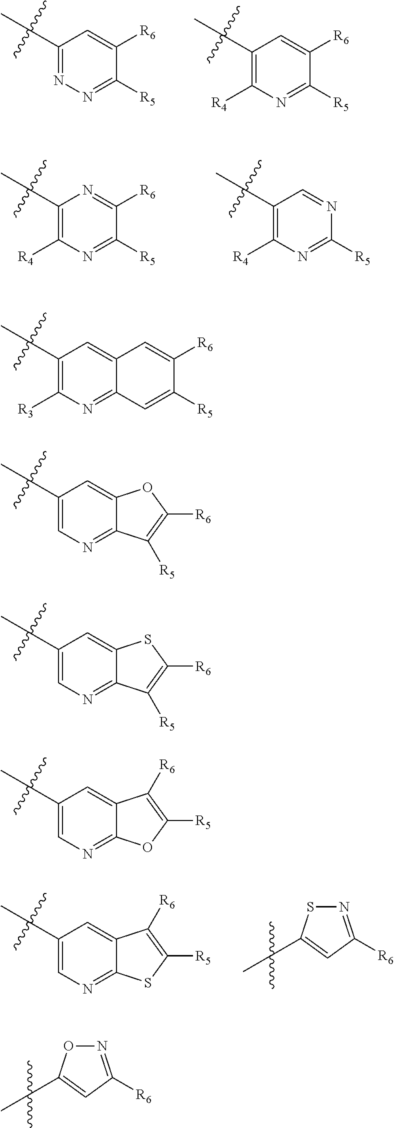

R₄ is selected from the group consisting of hydrogen, alkyl, and halogen; R₅ is selected from the group consisting of hydrogen, alkyl, halogen, nitro, and —NR₁₀R₁₁, wherein R₁₀ and R₁₁ are independently selected from the group consisting of hydrogen and lower alkyl; R₆ is selected from the group consisting of hydrogen, alkenyl, alkoxy, alkoxyalkoxy, alkoxyalkyl, alkoxycarbonyl, alkoxycarbonylalkyl, alkyl, alkylcarbonyl, alkylcarbonyloxy, alkylthio, alkynyl, amino, aminoalkyl, aminocarbonyl, aminocarbonylalkyl, aminosulfonyl, carboxy, carboxyalkyl, cyano, cyanoalkyl, formyl, formylalkyl, haloalkoxy, haloalkyl, halogen, hydroxyl, hydroxyalkyl, mercapto, mercaptoalkyl, nitro, 5-tetrazolyl, —NR₇SO₂R₈, —C(NR₇)NR₈R₉, —CH₂C(NR₇)NR₈R₉, —C(NOR₇)R₈, —C(NCN)R₇, —C(NNR₇R₈)R₉, —S(O)₂OR₇, and —S(O)₂R₇; and R₇, R₈, and R₉ are independently selected from the group consisting of hydrogen and alkyl; provided that when R₃ is pyridazine then R₁ is alkyl.

Representative compounds of formula (viii) include, but are not limited to: N-[(3S)-1-(6-chloro-3-pyridinyl)pyrrolidinyl]-N-methylamine; (3S)-1-(6-chloro-3-pyridinyl)pyrrolidinylamine; N-[(3S)-1-(6-chloro-3-pyridinyl)pyrrolidinyl]-N,N-dimethylamine; (3R)-1-(6-chloro-3-pyridinyl)pyrrolidinylamine; N-[(3R)-1-(6-chloro-3-pyridinyl)pyrrolidinyl]-N-methylamine; N-[(3R)-1-(6-chloro-3-pyridinyl)pyrrolidinyl]-N,N-dimethylamine; 1-(6-chloro-3-pyridinyl)-3-pyrrolidinylamine; (3S)-1-(3-pyridinyl)pyrrolidinylamine; N-methyl-N-[(3S)-1-(3-pyridinyl)pyrrolidinyl]amine; 1-(3-pyridinyl)-3-pyrrolidinylamine; (3R)-1-[5-(trifluoromethyl)-3-pyridinyl]pyrrolidinylamine; N-methyl-N-{(3R) 1-[5-(trifluoromethyl)-3-pyridinyl]pyrrolidinyl}amine; (3S)-1-[5-(trifluoromethyl)-3-pyridinyl]pyrrolidinylamine; N-methyl-N-{(3S)-1-[5-(trifluoromethyl)-3-pyridinyl]pyrrolidinyl}amine; (3R)-1-(6-chloro-5-chloro-5-methyl-3-pyridinyl)pyrrolidinyl amine; N-[(3R)-1-(6-chloro-5-methyl-3-pyridinyl)pyrrolidinyl]-N-methylamine; (3S)-1-(6-chloro-5-methyl-3-pyridinyl)pyrrolidinylamine; N-[(3S)-1-(6-chloro-5-methyl-3-pyridinyl)pyrrolidinyl]-N-methylamine; (3S)-1-(5,6-dichloro-3-pyridinyl)pyrrolidinylamine; N-[(3S)-1-(5,6-dichloro-3-pyridinyl)pyrrolidinyl]-N-methylamine; (3R)-1-(5,6-dichloro-3-pyridinyl)pyrrolidinylamine; N-[(3R)-1-(5,6-dichloro-3-pyridinyl)pyrrolidinyl]-N-methylamine; (3S)-1-(6-chloro-5-methoxy-3-pyridinyl)pyrrolidinylamine; N-[(3S)-1-(6-chloro-5-methoxy-3-pyridinyl)pyrrolidinyl]-N-methylamine; (3S)-1-(6-fluoro-5-methyl-3-pyridinyl)pyrrolidinylamine; N-[(3S)-1-(6-fluoro-5-methyl-3-pyridinyl)pyrrolidinylamine; N-[(3S)-1-(6-fluoro-5-methyl-3-pyridinyl)pyrrolidinyl]-N-methylamine; (3R)-1-(6-fluoro-5-methyl-3-pyridinyl)pyrrolidinylamine; N-[(3R)-1-(6-fluoro-5-methyl-3-pyridinyl)pyrrolidinyl]-N-methylamine; (3S)-1-(5-nitro-3-pyridinyl)pyrrolidinylamine; N-methyl-N-[(3S)-1-(5-nitro-3-pyridinyl)pyrrolidinyl]amine; (3R)-1-(5-nitro-3-pyridinyl)pyrrolidinylamine; N-methyl-N-[(3R)-1-(5-nitro-3-pyridinyl)pyrrolidinyl]amine; and (2S,3R)-2-(chloromethyl) 1-(3-pyridinyl)pyrrolidinylamine. Compounds corresponding to formula (viii) are described in further detail in International Publication WO 00/71534; and U.S. Pat. No. 6,833,370 (each of which is hereby incorporated by reference herein in its entirety).

In another particular embodiment, the compound is an indazole, benzothioazole, or benzoisothiazole. According to this embodiment, for example, the compound administered to the patient may have the formulae (ix)(a), (ix)(b), (ix)(c), or (ix)(d):

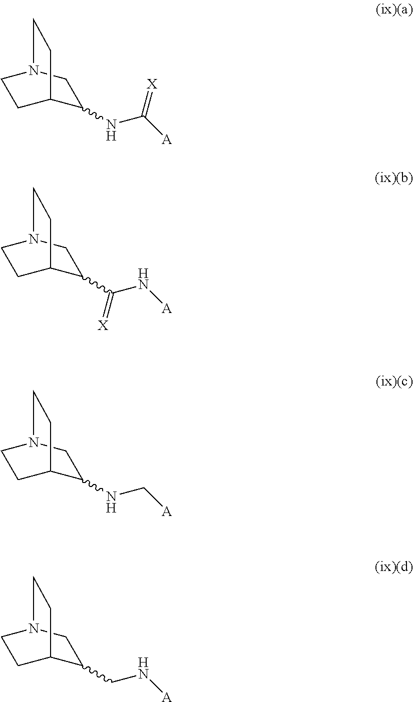

wherein A is:

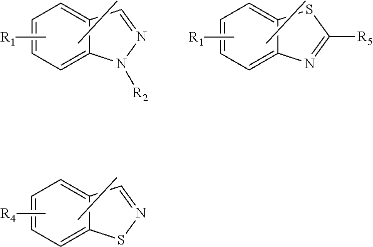

wherein the slanted line through the fused rings represent the bond of attachment from the fused chemical moiety to the remainder of the compound; X is O or S; R₁ is H, F, Cl, Br, I, OH, CN, nitro, NH₂, alkyl having 1 to 4 carbon atoms, fluorinated alkyl having 1 to 4 carbon atoms (e.g., CF₃), cycloalkyl having 3 to 7 carbon atoms, cycloalkylalkyl having 4 to 7 carbon atoms, alkoxy having 1 to 4 carbon atoms (e.g., OCH₃), cycloalkoxy having 3 to 7 carbon atoms, cycloalkylalkoxy having 4 to 7 carbon atoms, alkylthio having 1 to 4 carbon atoms (e.g., SCH₃), fluorinated alkoxy having 1 to 4 carbon atoms (e.g., OCF₃, OCHF₂), hydroxyalkyl having 1 to 4 carbon atoms, hydroxyalkoxy having 2 to 4 carbon atoms, monoalkylamino having 1 to 4 carbon atoms, dialkylamino wherein each alkyl group independently has 1 to 4 carbon atoms, Ar or Het; R₂ is H, alkyl having 1 to 4 carbon atoms, cycloalkyl having 3 to 7 carbon atoms, or cycloalkylalkyl having 4 to 7 carbon atoms; R₃ is H, F, Cl, Br, I, OH, CN, nitro, NH₂, alkyl having 1 to 4 carbon atoms, fluorinated alkyl having 1 to 4 carbon atoms (e.g., CF₃), cycloalkyl having 3 to 7 carbon atoms, cycloalkylalkyl having 4 to 7 carbon atoms, alkoxy having 1 to 4 carbon atoms (e.g., OCH₃), cycloalkoxy having 3 to 7 carbon atoms, cycloalkylalkoxy having 4 to 7 carbon atoms, alkylthio having 1 to 4 carbon atoms (e.g., SCH₃), fluorinated alkoxy having 1 to 4 carbon atoms (e.g., OCF₃, OCHF₂), hydroxyalkyl having 1 to 4 carbon atoms, hydroxyalkoxy having 2 to 4 carbon atoms, monoalkylamino having 1 to 4 carbon atoms, dialkylamino wherein each alkyl group independently has 1 to 4 carbon atoms, Ar or Het; R₄ is H, F, Cl, Br, I, OH, CN, nitro, NH₂, alkyl having 1 to 4 carbon atoms, fluorinated alkyl having 1 to 4 carbon atoms (e.g., CF₃), cycloalkyl having 3 to 7 carbon atoms, cycloalkylalkyl having 4 to 7 carbon atoms, alkoxy having 1 to 4 carbon atoms (e.g., OCH₃), cycloalkoxy having 3 to 7 carbon atoms, cycloalkylalkoxy having 4 to 7 carbon atoms, alkylthio having 1 to 4 carbon atoms (e.g., SCH₃), fluorinated alkoxy having 1 to 4 carbon atoms (e.g., OCF₃, OCHF₂), hydroxyalkyl having 1 to 4 carbon atoms, hydroxyalkoxy having 2 to 4 carbon atoms, monoalkylamino having 1 to 4 carbon atoms, dialkylamino wherein each alkyl group independently has 1 to 4 carbon atoms, Ar or Het; R₅ is H, F, Cl, Br, I, OH, CN, nitro, NH₂, alkyl having 1 to 4 carbon atoms, fluorinated alkyl having 1 to 4 carbon atoms (e.g., CF₃), cycloalkyl having 3 to 7 carbon atoms, cycloalkylalkyl having 4 to 7 carbon atoms, alkoxy having 1 to 4 carbon atoms (e.g., OCH₃), cycloalkoxy having 3 to 7 carbon atoms, cycloalkylalkoxy having 4 to 7 carbon atoms, alkylthio having 1 to 4 carbon atoms (e.g., SCH₃), fluorinated alkoxy having 1 to 4 carbon atoms (e.g., OCF₃, OCHF₂), hydroxyalkyl having 1 to 4 carbon atoms, hydroxyalkoxy having 2 to 4 carbon atoms, monoalkylamino having 1 to 4 carbon atoms, dialkylamino wherein each alkyl group independently has 1 to 4 carbon atoms, Ar or Het; Ar is an aryl group containing 6 to 10 carbon atoms which is unsubstituted or substituted one or more times by alkyl having 1 to 8 carbon atoms, alkoxy having 1 to 8 carbon atoms, halogen (F, Cl, Br, or I, preferably F or Cl), dialkylamino wherein the alkyl portions each have 1 to 8 carbon atoms, amino, cyano, hydroxyl, nitro, halogenated alkyl having 1 to 8 carbon atoms, halogenated alkoxy having 1 to 8 carbon atoms, hydroxyalkyl having 1 to 8 carbon atoms, hydroxyalkoxy having 2 to 8 carbon atoms, alkenyloxy having 3 to 8 carbon atoms, alkylthio having 1 to 8 carbon atoms, alkylsulphinyl having 1 to 8 carbon atoms, alkylsulphonyl having 1 to 8 carbon atoms, monoalkylamino having 1 to 8 carbon atoms, cycloalkylamino wherein the cycloalkyl group has 3 to 7 carbon atoms and is optionally substituted, aryloxy wherein the aryl portion contains 6 to 10 carbon atoms (e.g., phenyl, naphthyl, biphenyl) and is optionally substituted, arylthio wherein the aryl portion contains 6 to 10 carbon atoms (e.g., phenyl, naphthyl, biphenyl) and is optionally substituted, cycloalkyloxy wherein the cycloalkyl group has 3 to 7 carbon atoms and is optionally substituted, sulfo, sulfonylamino, acylamido (e.g., acetamido), acyloxy (e.g., acetoxy) or combinations thereof; and Het is a heterocyclic group, which is fully saturated, partially saturated or fully unsaturated, having 5 to 10 ring atoms in which at least 1 ring atom is a N, O or S atom, which is unsubstituted or substituted one or more times by halogen (F, Cl, Br, or I, preferably F or Cl), aryl having 6 to 10 carbon atoms (e.g., phenyl, naphthyl, biphenyl) and is optionally substituted, alkyl having 1 to 8 carbon atoms, alkoxy having 1 to 8 carbon atoms, cyano, trifluoromethyl, nitro, oxo, amino, monoalkylamino having 1 to 8 carbon atoms, dialkylamino wherein each alkyl group has 1 to 8 carbon atoms, or combinations thereof; and pharmaceutically acceptable salts thereof.

Compounds corresponding to formulae (ix)(a), (ix)(b), (ix)(c), and (ix)(d) are described in further detail in U.S. Pat. No. 7,429,664 (which is hereby incorporated by reference herein in its entirety)

In general, for the compounds of formulae (i), (ii), (iii), (iv), (v), (vi), (vii), (viii), and (ix)(a-d), definitions for the chemical moieties recited in the various substituent groups are the same as those found in the patent or published application cited above in connection with the formulae. Also, with regard to stereoisomers, it should be understood that a solid line designation for the bonds in the compositions corresponding to formulae (i), (ii), (iii), (iv), (v), (vi), (vii), (viii), and (ix)(a-d) (and any others herein) for attachment of an substituent group to a chiral carbon atom of the compound indicates that these groups may lie either below or above the plane of the page (i.e., [symbol] or [symbol] ) All isomeric forms of the compounds disclosed herein are contemplated, including racemates, racemic mixtures, and individual enantiomers or diastereomers.

Another compound that may be used in the methods described herein is anabaseine, i.e., 2-(3-pyridyl)-3,4,5,6-tetrahydropyridine which is a naturally occurring toxin in certain marine worms (nemertine worms) and ants (see, e.g., Kem et al., Toxicon, 9:23, 1971) and is a potent activator of mammalian nicotinic receptors (see, e.g., Kem, Amer. Zoologist, 25, 99, 1985). Certain anabaseine analogs may also be employed, such as DMAB (3-[4-(dimethylamino)benzylidene]-3,4,5,6-tetrahydro-2′,3′-bipyridin-e) (see, e.g., U.S. Pat. No. 5,602,257 and WO 92/15306 (each of which is hereby incorporated by reference herein), and (E-3-[2,4-dimethoxy-benzylidene]-anabasine, also known as GTS-21 and DMXB (see, e.g., U.S. Pat. No. 5,741,802 and U.S. Pat. No. 5,977,144 (each of which is hereby incorporated by reference herein in its entirety). Another compound that may be used tropisetron, i.e., 1αH, 5αH-tropan-3α-yl indole-3-carboxylate (see J. E. Macor et al., Bioorg. Med. Chem. Lett. 2001, 319-321).

Still other compounds having nicotinic acetylcholine receptor activity that may be used in the methods of the present disclosure include, for instance, U.S. Published Patent Application No. 2002/00288809; U.S. Published Patent Application No. 2009/0012127; U.S. Pat. No. 6,303,638; U.S. Pat. No. 6,846,817; U.S. Pat. No. 7,244,745; and U.S. Pat. No. 7,429,664 (each of which is hereby incorporated by reference herein).

Improving Peripheral Nerve Sensory Loss

Also provided are methods for improving a symptom of peripheral nerve sensory loss in a patient. In general, the methods comprise administering to a patient exhibiting the symptom(s) a compound having nicotinic acetylcholine receptor activity, such as those described above. In one embodiment, the compound is selected from the group consisting of ABT-089, ABT-894, alpha-bungarotoxin, anabaseine, bupropion, buspirone, BW284c51, cytisine, dihydro-beta-erythoidine, DMXB, DMXB-A (GTS-21), diazoxon, donepezil, exelon, fluoxetine, galantamine, huperzine A, ispronicline (TC-1734/AZD-3480), lobeline, mecamylamine, MEM3454, MEM63908, methyllycaconitine, nefazodone, octanol/ethanol, OmIA, paroxetine, sertraline, tacrine, TC-2559, TC-5214 ((S)-(+)-mecamylamine), TC-5619, tebanicline (ABT-594), varenicline, venlafaxine, XY4083, and combinations thereof. In one another embodiment, the compound is selected from the group consisting of ABT-089, ABT-894, bupropion, cytisine, dianicline (SSR591813), DMXB-A (GTS-21), ispronicline (TC-1734/AZD-3480), lobeline, mecamylamine, methyllycaconitine, TC-2559, TC-5214 ((S)-(+)-mecamylamine), tebanicline (ABT-594), varenicline, and combinations thereof. In another embodiment, the compound is selected from the group consisting of donepezil, exelon, fluoxetine, galantamine, huperzine A, MEM3454, MEM63908, tacrine, XY4083, and combinations thereof. In one preferred embodiment, the compound is varenicline.

Generally, the methods involve first diagnosing or assessing the level of the peripheral nerve sensory loss symptom in the patient to provide a baseline level or measurement of the symptom (e.g., by virtue of its severity or intensity). Thereafter, the patient is administered (i.e., is treated with) a compound having nicotinic acetylcholine receptor activity, typically in the form of a pharmaceutical composition comprising the compound and a pharmaceutically acceptable carrier.

At some point during or after administration of the compound (e.g., 5 minutes, 10 minutes, 15 minutes, 30 minutes, 1 hour, 3 hours, 6 hours, 12 hours, 18 hours, 24 hours, 36 hours, 48 hours, or 72 hours, or longer (e.g., weeks, months, etc.), the patient's symptoms are again diagnosed or assessed. That is, a second measurement or level of the symptoms is taken; this measurement may be designated as a midpoint level or an endpoint level, depending on whether or not more treatments (i.e., further administrations of the compound) and/or symptom assessments are contemplated. The second (or subsequent) measurement may be compared to the baseline measurement to evaluate the efficacy of the treatment. Preferably, the second (or subsequent) measurement (as a midpoint or endpoint) taken after administration of the compound is improved relative to the baseline measurement.

This treatment and assessment regime may be repeated as many times as desired, with second, third, fourth, fifth, and so on, measurements being compared to the original baseline measurement taken prior to administration of the compound or otherwise initiating treatment. Assessments can be taken while the patient is still on a treatment regime (i.e., during the period of time that the patient is given the compound and while it is present in their system), and assessments may also be taken after a patient has stopped treatment and/or after complete washout or elimination of the compound from the patient's system.

Various scales and tests can assess symptoms of peripheral nerve sensory loss in a patient and the effect of the compounds described herein on the treatment of the symptom. These are, for example, and without limitation, monofilament testing (such as those employing the Semmes-Weinstein (SW) monofilament test devices and methods, and/or those described in U.S. Pat. No. 5,316,011; U.S. Pat. No. 5,381,806; and U.S. Pat. No. 6,196,976; see also, Frykberg et al., Diabetic Foot Disorders: A Clinical Practice Guideline, J. Foot Ankle Surg. 2006; 45(5): S2-66); electromyogram (EMG) and nerve conduction studies (see, e.g., Reaz et al., Biological Procedures Online, vol. 8, issue 1, pp. 11-35, March 2006; Kleissen et al., Gait Posture. 1998; 8(2):143-158; Pagana K D, Pagana T J (2006). Mosby's Manual of Diagnostic and Laboratory Tests, 3rd ed. St. Louis: Mosby; somatosensory evoked potentials (i.e., a series of waves that reflect sequential activation of neural structures along the somatosensory pathways) (see, e.g., Chiappa et al., Evoked Potentials in Clinical Medicine, pages 283-428 (Lippincott Williams & Wilkins, 3rd ed. (1997))); nerve biopsy (i.e., removal of a small piece of a nerve for examination, such as from the sural nerve or superficial radial nerve); standard neurologic examinations (e.g., pin prick, vibratory sense using tuning fork, light touch sensation (brush), position sense, stereognosia, graphestheia, extinction); and the Friedreich's Ataxia Rating Scale (FARS) (see, e.g., Subramony et al., Neurology. 2005. 64(7):1261-2, modified by Lynch et al., Neurology. 2006. 66(11):1711-6. Some of these and other ratings scales are described in Herndon, Handbook of Neurologic Rating Scales (2nd ed. 2006).

These scales or measures generally are carried out by performing a mechanical examination on the patient (e.g., manipulating the patient's extremities with a device, such as a monofilament or brush tests, light pinpricks, etc.) and assigning a score based on the intensity or frequency of the symptoms and the ability, partial ability, or inability of the patient to respond to the stimuli or to perform various tasks. The scales may also target or focus upon improvements in symptoms since a previous assessment. In certain embodiments, a total or overall score on the assessment or scale is calculated. If desired, multiple scales or tests can be administered and their results combined. Typically, a baseline score is compared to a second, subsequent (midpoint or endpoint) score to determine the change in severity or frequency of peripheral nerve sensory loss after treatment with the compound(s) described herein.

Once the compound has been administered, a patient's symptoms may remain improved relative to the baseline level, even after treatment has ceased and no further administrations of the compound a performed. The patient's symptoms may remain at an improved level, for example, for 1 day, 3 days, 5 days, 7 days, 3 weeks, 1 month, 3 months, 6 months, 1 year, or longer, after the final administration of the compound. In this regard, the methods described herein can be said to beneficially alter the chemical and/or biochemical pathways of the patient.

Administration and Mode of Treatment

In general, where the pharmaceutical agents discussed above are administered to a patient in need of treatment of one or more of the symptoms noted above, the agent is administered in an effective amount; that is, an amount to achieve a therapeutic benefit.

In general, the compound having nicotinic acetylcholine receptor activity is administered to the patient in the form of a pharmaceutical composition or pharmaceutical formulation comprising the compound. The compositions or formulations generally comprise at least one active pharmaceutical ingredient having nicotinic acetylcholine receptor activity and a pharmaceutically acceptable carrier (discussed in further detail below). The structure and synthesis of many nicotinic acetylcholine receptor-active compounds are well known to persons of skill in the art. A description of several representative compounds is provided above, and may also be found in the patent and other literature. This includes, for example, the patents and published applications cited herein, each of which is hereby incorporated by reference herein in its entirety.

The dose or amount of the pharmaceutical agent administered to the patient should be an effective amount for the intended purpose; i.e., treatment of one or more of the symptoms discussed above. Generally speaking, the effective amount of the agent administered to the patient can vary according to a variety of factors such as, for example, the age, weight, sex, diet, route of administration, and the medical condition of the patient. Specifically preferred doses are discussed more fully below, or are provided on the label of the pharmaceutical agent(s) being administered, or is within the ambit of one skilled in the art. It will be understood that the total daily usage of the compounds discussed herein will be decided by the attending physician within the scope of sound medical judgment.

The specific therapeutically effective dose level for any particular patient will depend upon a variety of factors including the disease, pathological disorder, or medical condition of the patient, and the particular symptoms being treated and the severity of the same; activity of the specific composition(s) employed; the age, body weight, general health, sex and diet of the patient; the time of administration; the route of administration; the rate of excretion of the specific composition(s) employed; the duration of the treatment; drugs used in combination or coincidental with the specific composition(s) employed and like factors are well known in the medical arts. For example, it is well within the skill of the art to start doses of the compositions(s) at levels lower than those required to achieve the desired effect, and to gradually increase the dosage until the desired effect is achieved. By way of another example, the dose level can be gradually or abruptly decreased to minimize undesired side effects of the compound being administered. If desired, the effective daily doses may be divided into multiple doses for purposes of administration. Consequently, single dose compositions may contain such amounts or submultiples to make up the daily dose.

Administration of the pharmaceutical agent can occur as a single event or over a time course of treatment. For example, one or more of the compositions can be administered hourly (e.g., every hour, every two hours, every three hours, every four hours, every five hours, every six hours, and so on), daily, weekly, bi-weekly, or monthly. For treatment of acute conditions, the time course of treatment may be at least several hours or days. Certain conditions could extend treatment from several days to several weeks. For example, treatment could extend over one week, two weeks, or three weeks. For more chronic conditions, treatment could extend from several weeks to several months, a year or more, or the lifetime of the patient in need of such treatment. Alternatively, the compositions can be administered hourly, daily, weekly, bi-weekly, or monthly, for a period of several weeks, months, years, or over the lifetime of the patient. The pharmaceutical compositions may be administered to a patient on an empty stomach, or administered along with (i.e., before, during, or after) meals.

Dosage levels for the active agents are generally those indicated on the label of the pharmaceutical. One or more of the compounds may be utilized in a pharmaceutically acceptable carrier, additive or excipient at a suitable dose level ranging, for example, from about 0.05 to about 200 mg/kg of body weight per day, preferably within the range of about 0.1 to 100 mg/kg/day, most preferably in the range of 0.25 to 50 mg/kg/day. As noted above, the desired dose may conveniently be presented in a single dose or as divided doses administered at appropriate intervals, for example as two, three, four or more sub-doses per day.

Ideally, the active ingredient should be administered to achieve effective peak plasma concentrations of the active compound within the range of from about 0.05 uM to about 5 uM. Oral dosages, where applicable, will depend on the bioavailability of the compositions from the GI tract, as well as the pharmacokinetics of the compositions to be administered. For intravenous use, these concentrations may be achieved, for example, by the intravenous injection of about a 0.05 to 10% solution of the active ingredient, optionally in saline, or orally administered as a bolus containing about 1 mg to about 5 g, preferably about 5 mg to about 500 mg of the active ingredient, depending upon the active compound and its intended target. Desirable blood levels may be maintained by a continuous infusion to preferably provide about 0.01 mg/kg/hour to about 2.0 mg/kg/hour or by intermittent infusions containing about 0.05 mg/kg to about 15 mg/kg of the active ingredient. Likewise, continuous (e.g., hourly or daily) oral administration may be desired or necessary. While it is possible that, for use in therapy, one or more compositions of the invention may be administered as the raw chemical, it is preferable to present the active ingredient as a pharmaceutical formulation, presented in combination with a pharmaceutically acceptable carrier, excipient, or additive.

As noted above, the above-described compounds are generally dispersed in a pharmaceutically acceptable carrier prior to administration to the patient. The carrier, also known in the art as an excipient, vehicle, auxiliary, adjuvant, or diluent, is typically a substance which is pharmaceutically inert, confers a suitable consistency or form to the composition, and does not diminish the efficacy of the compound. The carrier is generally considered to be “pharmaceutically or pharmacologically acceptable” if it does not produce an unacceptably adverse, allergic or other untoward reaction when administered to a patient, especially a human.

The selection of a pharmaceutically acceptable carrier will also, in part, be a function of the route of administration. In general, the compositions can be formulated for any route of administration so long as the blood circulation system is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Typically, the route of administration is oral.

Pharmaceutically acceptable carriers for use in combination with the compounds described herein are well known to those of ordinary skill in the art and are selected based upon a number of factors: the particular compound used, and its concentration, stability and intended bioavailability; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2 to 30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)actamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di-, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di-, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄ to C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2 to 30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3 to 30 carbon atoms (e.g., acetone, methyl ethyl ketone, methyl isobutyl ketone); aliphatic, cycloaliphatic or aromatic hydrocarbons having 4 to 30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1 to 30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in formulations are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), and A. J. Spiegel et al., Use of Nonaqueous Solvents in Parenteral Products, Journal of Pharmaceutical Sciences, Vol. 52, No. 10, pp. 917-927 (1963).

Formulations containing the active agents described above may take the form of solid, semi-solid, lyophilized powder, or liquid dosage forms such as, for instance, aerosols, capsules, creams, emulsions, foams, gels/jellies, lotions, ointments, pastes, powders, soaps, solutions, sprays, suppositories, suspensions, sustained-release formulations, tablets, tinctures, transdermal patches, and the like, preferably in unit dosage forms suitable for simple administration of precise dosages. Typically, the active agent is administered in tablet or pill form, including, for example, soft chewable tablets, hard chewable tablets, and hard swallowable tablets; various sizes and shapes of tablets may be formed, generally by varying the size and shape of the die and punch. Representative tablet shapes include briquette, circular (i.e., cylindrical), lozenge, and pillow shapes. The size and shape of the tablet may depend, in part, on the various components in the tablet and their amounts relative to other components in the tablet.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

OVERVIEW: Two patients with atypical Friedreich ataxia (heterozygotes for a GAA expansion and a G130V point mutation) experienced modest proprioceptive improvements in their extremities within a month of taking varenicline (Chantix®), a drug approved for smoking cessation.

Introduction:

Friedreich ataxia (FA) is a progressive, neurodegenerative disease that is characterized by gait and limb ataxia, imbalance, dysarthria, areflexia, and loss of position sense. FA is the most common cause of inherited ataxia (1), affecting approximately 1 to 2/50,000 people. Medications that effectively treat the symptoms of FA are currently lacking. The majority of FA patients are homozygous for GAA trinucleotide repeat expansions in the first intron of the FXN gene (2). However, 2-5% of patients are compound heterozygotes for a GAA expansion and a frataxin point mutation (3). One mutation, the G130V mutation, is commonly associated with a less severe phenotype and preservation of reflexes. In this report, we present the histories of 2 brothers who were heterozygous for GAA trinucleotide repeat expansion and G130V point mutations. Both brothers experienced modest improvements in lower extremity proprioception and upright posture shortly after taking varenicline, (Chantix®), a drug used for smoking cessation.

The patients (aged 54 and 50) developed symptoms of FA, including lower extremity weakness and gait dysfunction, at approximately 18 years of age. Both brothers and their sister were found to be heterozygotes for GAA expansion and G130V point mutations (840 repeats in the brothers, and 875 repeats in their sister). Their symptoms included progressive weakness, ataxia, and proprioceptive dysfunction, although they retained the ability to walk with assistance. Their past medical histories included diabetes and normal cardiac function. Neither brother had a history of smoking. One brother had been treated with idebenone in the past, but discontinued it several years ago, but neither brother used any additional neurotransmitter-related drugs. Cardiac tests, including electrocardiograms and echocardiograms that were performed within 6 months of their visit, were normal.

The brothers presented to an academic movement disorders center for evaluation. The eldest brother had normal vital signs, mini-Mental State Exam (MMSE) and cranial nerves. His feet were disfigured and everted due to multiple fractures caused by severely impaired sensation. Motor examinations revealed moderate to severe atrophy in the lower extremities, but relative preservation of muscle mass in the upper extremities. Severe distal weakness was noted in his lower extremities, with 1/5 strength bilaterally in his distal leg muscles and 3+/5 strength in his bilateral proximal leg muscles. He was unable to feel light or deep touch or vibration in his feet up to his mid-calf bilaterally, and could not discern which foot or toe was being touched while his eyes were closed. Reflexes could not be elicited in his lower extremities. Tests for cerebellar function could not be performed in his legs due to severe weakness, but there was no dysmetria in the upper extremities. The patient was able to stand without the help of a walker for only 2 seconds. He needed the assistance of a walker for ambulation, and completed a timed 25-foot walk in 15 seconds. The scale for the assessment and rating of ataxia (SARA) was 24.

The younger brother presented with normal vital signs, MMSE and cranial nerves. His motor examinations also revealed moderate to severe atrophy in the lower extremities, but relative preservation of muscle mass in the upper extremities. He had normal strength in his upper extremities, but 4−/5 weakness in his proximal lower extremities, and 1/5 strength in his distal lower extremities. The patient had a severe loss of light touch in his feet up to his mid-calf bilaterally. Vibration and position sense were severely diminished in his lower extremities and moderately decreased in his upper extremities. The patient had areflexia in his lower extremities, and trace reflexes in his upper extremities. Tests for cerebellar function could not be performed in his legs due to severe weakness, and there was no dysmetria in the upper extremities. He could stand without holding on to his walker for 23 seconds, and completed the timed 25-foot walk in 21 seconds with the assistance of a walker. His SARA score was 23.

Both patients were started on varenicline 0.5 mg once a day, with dose increases by 0.5 mg every 4 days until a maximum dose of 1 mg twice daily was reached. Fourteen days after starting varenicline, the eldest brother's strength was unchanged. However, he reported he became able to feel a touch on his legs for the first time in many years, and that he now had a “sense of where his feet were”. After 21 days, the he could stand with his feet apart unassisted for up to 30 seconds while performing a rhythmic “patty-cake” maneuver, in which he alternately clapped his hands together and then on his thighs. He also noted improved upright posture. Thirty days after starting varenicline, he could correctly tell which of his feet and toes were being touched, and his 25-foot timed walk improved to 10 seconds, an improvement of 5 seconds (33%). The SARA scale was reduced by 1 point due to the improvement in the patient's ability to stand in a natural position without support. He was weaned off varenicline 7 weeks after starting it. However, he noted continued improvement in proprioception and posture for 4 to 6 weeks after stopping the drug, after which his exam reverted to baseline subjectively and objectively.

His younger brother similarly reported improved sensation in his hands and legs 14 to 21 days after taking the maximum varenicline dose of 1 mg twice daily. He noted that he could zip his pants without having to look down to assist in the task, could tell which toe and foot was being touched and had better upright posture. He had mildly improved posture post-treatment, but unchanged gait. The patient weaned off varenicline, and found that his examination returned to baseline subjectively after 4 to 6 weeks. Neither brother had any significant side effects from varenicline.

There is currently no pharmacologic treatment of FA. Several drugs have been reported to have some benefit in improving symptoms of FA, including amantadine hydrochloride (4), 5-hydroxytryptophan (5), vigabatrin (6), and physostigmine (7). However, these medications have shown no consistent, significant improvement in FA symptoms. Varenicline is a highly selective partial agonist at α4β2 nicotinic acetylcholine receptors, and a full agonist at α7 nicotinic receptors (8). The exact mechanism of action for the possible beneficial effect of varenicline in these cases is unknown. Dorsal root ganglion (DRG) neurons express several nicotinic acetylcholine receptor (nAChR) subunits, including α 3., α 4., α 5., α 7., α 9, and α 10, and convey sensory information from the peripheral to the central nervous system. The nicotinic receptors are selectively concentrated in smaller DRG cells that are not traditionally associated with proprioceptive transmission. FA selectively affects large neurons of the dorsal root ganglia (9).

Further investigation is needed to confirm these observations in a blinded fashion in groups of patients with FA. The potential mechanisms for a possible beneficial effect of varenicline in FA is unclear, but speculative mechanisms might include a possible facilitation of action potential propagation through surviving sensory axons, or the enhanced activation of alternative, typically subliminal sensory pathways to restore proprioception and other sensory modalities.

### REFERENCES

1. Cossee M, Schmitt M, Campuzano V, et al. Evolution of the Friedreich's ataxia trinucleotide repeat expansion: founder effect and premutations. Proc. Natl. Acad. Sci. 1997; 94:7452-57.
2. Campuzano V, Montermini L, Molto M D, et al. Friedreich's ataxia autosomal recessive disease caused by an intronic GAA triplet repeat expansion. Science 1996. 271:1423-27.
3. Cossée M, Dürr A, Schmitt M, et al. Friedreich's ataxia: point mutations and clinical presentation of compound heterozygotes. Ann Neurol. 1999 February; 45(2):200-6.
4. Peterson P L, Saad J, Nigro M A. The treatment of Friedreich's ataxia with amantadine hydrochloride. Neurology. 1988 September; 38(9):1478-80.
5. Trouillas P, Serratrice G, Laplane D, et al. Levorotatory form of 5-hydroxytryptophan in Friedreich's ataxia. Results of a double-blind drug-placebo cooperative study. Arch Neurol. 1995 May; 52(5):456-60.
6. De Smet Y, Mear J Y, Tell G, Schechter P H, Lhermitte F, Agid Y. Effect of gamma-vinyl GABA in Friedreich's ataxia. Can J Neurol Sci. 1982 May; 9(2):171-3.
7. Kark R A, Budelli M M, Wachsner R. Double-blind, triple-crossover trial of low doses of oral physostigmine in inherited ataxias. Neurology. 1981 March; 31(3):288-92.
8. Mihalak K B, Carroll F I, Luetje C W. Varenicline is a partial agonist at alpha4beta2 and a full agonist at alpha7 neuronal nicotinic receptors. Mol Pharmacol. 2006 September; 70(3):801-5.
9. Inoue K, Hirano A, Hasson J. Friedreich's ataxia selectively involves the large neurons of the dorsal root ganglia. Trans Am Neurol Assoc. 1979; 104:75-6.

1. A method of treating peripheral nerve sensory loss in a human, the method comprising administering to the human a compound having nicotinic acetylcholine receptor activity.

2. The method of claim 1 further comprising
determining a baseline measurement of peripheral nerve sensory loss in the human and thereafter administering to the human the compound having nicotinic acetylcholine receptor activity; and
determining a second measurement of peripheral nerve sensory loss in the human during or after administration of the compound, wherein an improvement in the second measurement relative to the baseline measurement indicates treatment of the peripheral nerve sensory loss.

3. The method of claim 1 further comprising
determining a baseline measurement of peripheral nerve sensory loss in the human and thereafter administering to the human the compound having nicotinic acetylcholine receptor activity; and
determining a second measurement of peripheral nerve sensory loss in the human at least one month after administration of the compound has ceased;
wherein the second measurement is improved relative to the baseline measurement.

4. The method of claim 1
wherein a second measurement of peripheral nerve sensory loss measured after ceasing administration of the compound is improved relative to a baseline measurement of peripheral nerve sensory loss measured prior to administration of the compound.

5. The method of claim 1 wherein the compound is a smoking cessation agent.

6. The method of claim 1 wherein the compound is selected from the group consisting of (i) an aryl-fused azapolycyclic compound; (ii) a pyridopyranoazepine; (iii) an aryl-substituted olefinic amine compound; (iv) a benzylidene- or cinnamylidene-anabaseine compound; (v) a heterocyclic ether compound; (vi) 3-pyridyloxyalkyl heterocyclic ether compound; (vii) an N-substituted diazabicyclic compound; (viii) a heterocyclic substituted amino azacycle compound; and (ix) an indazole, benzothioazole, or benzoisothiazole compound.

7. The method of claim 1 wherein the compound is selected from the group consisting of: ABT-089, ABT-894, alpha-bungarotoxin, anabaseine, bupropion, buspirone, BW284c51, cytisine, dianicline (SSR591813), dihydro-beta-erythoidine, DMXB, DMXB-A (GTS-21), diazoxon, donepezil, exelon, fluoxetine, galantamine, huperzine A, ispronicline (TC-1734/AZD-3480), lobeline, mecamylamine, MEM3454, MEM63908, methyllycaconitine, nefazodone, octanol/ethanol, OmIA, paroxetine, sertraline, tacrine, TC-2559, TC-5214 ((S)-(+)-mecamylamine), TC-5619, tebanicline (ABT-594), varenicline, venlafaxine, XY4083, and combinations thereof.

8. The method of claim 1 wherein the compound is selected from the group consisting of ABT-089, ABT-894, bupropion, cytisine, dianicline (SSR591813), DMXB-A (GTS-21), ispronicline (TC-1734/AZD-3480), lobeline, mecamylamine, methyllycaconitine, TC-2559, TC-5214 ((S)-(+)-mecamylamine), tebanicline (ABT-594), varenicline, and combinations thereof.

9. The method of claim 1 wherein the compound is selected from the group consisting of donepezil, exelon, fluoxetine, galantamine, huperzine A, MEM3454, MEM63908, tacrine, XY4083, and combinations thereof.

10. The method of claim 1 wherein the compound is varenicline.

11-14. (canceled)

15. The method of claim 5 wherein the compound is varenicline.

16. (canceled)

17. (canceled)

18. The method of claim 6 wherein the aryl-fused azapolycyclic compound has the formula (i): 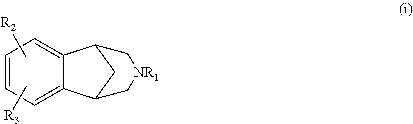
wherein R₁ is hydrogen, (C₁-C₆)alkyl, unconjugated (C₃-C₆)alkenyl, benzyl, XC(═O)R₁₃ or —CH₂CH₂—O—(C₁-C₄)alkyl;
R₂ and R₃ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino-, [(C₁-C₆)alkyl]₂-amino-, —CO₂R₄, —CONR₅R₆, —SO₂NR₇R₈, —C(═O)R₁₃, —XC(═O)R₁₃, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur, and X₂(C₀-C₆)alkoxy-(C₀-C₆)alkyl-, wherein X₂ is absent or X₂ is (C₁-C₆)alkylamino- or [(C₁-C₆)alkyl]₂amino-, and wherein the (C₀-C₆)alkoxy-(C₀-C₆)alkyl- moiety of said X₂(C₀-C₆)alkoxy-(C₀-C₆)alkyl- contains at least one carbon atom, and wherein from one to three of the carbon atoms of said (C₀-C₆)alkoxy-(C₀-C₆)alkyl- moiety may optionally be replaced by an oxygen, nitrogen or sulfur atom, with the proviso that any two such heteroatoms must be separated by at least two carbon atoms, and wherein any of the alkyl moieties of said (C₀-C₆)alkoxy-(C₀-C₆)alkyl- may be optionally substituted with from two to seven fluonne atoms, and wherein one of the carbon atoms of each of the alkyl moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluonne atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)—, [(C₁-C₆)alkyl]₂amino-, —CO₂R₄, —CONR₅R₆, —SO₂NR₇R₈, —C(═O)R₁₃ and —XC(═O)R₁₃; or R₂ and R₃, together with the carbons to which they are attached, form a four to seven membered monocyclic, or a ten to fourteen membered bicyclic, carbocyclic ring that can be saturated or unsaturated, wherein from one to three of the nonfused carbon atoms of said monocyclic rings, and from one to five of the carbon atoms of said bicyclic rings that are not part of the benzo ring shown in formula (i), may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂-amino-, —CO₂R₄, —CONR₅R₆, —SO₂NR₇R₈, —C(═O)R₁₃, and —XC(═O)R₁₃;
each R₄, R₅, R₆, R₇, R₈ and R₁₃ is selected, independently, from hydrogen and (C₁-C₆) alkyl, or R₅ and R₆, or R₇ and R₈ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and each X is, independently, (C₁-C₆)alkylene: with the proviso that: (a) at least one of R₁, R₂ and R₃ must be the other than hydrogen, and (b) when R₂ and R₃ are hydrogen, R₁ cannot be methyl or hydrogen; and the pharmaceutically acceptable salts of such compounds.

19. The method of claim 6 wherein the aryl-fused azapolycyclic compound is varenicline.

20. The method of claim 1 wherein the method is for treating peripheral nerve sensory loss resulting from Friedriech's ataxia.

21. The method of claim 15 wherein the method is for treating peripheral nerve sensory loss resulting from Friedriech's ataxia.

22. The method of claim 1 wherein the peripheral nerve sensory loss is disease-induced.

23. The method of claim 1 wherein the peripheral nerve sensory loss is drug- or toxin-induced.

24. The method of claim 1 wherein the peripheral nerve sensory loss is induced by acute or traumatic injury.

25. The method of claim 1 wherein the peripheral nerve sensory loss is the result of brain injury or spinal cord injury.
